(19) United States
(12) Patent Application Publication
(10) Pub. No.: US 2022/0175718 A1
(43) Pub. Date: Jun. 9, 2022

(54) TRANSDERMAL PHARMACEUTICAL FORMULATIONS FOR THE TREATMENT OF CANCER

(71) Applicant: PIKE THERAPEUTICS, INC., Vancouver (CA)

(72) Inventors: Fotios M. Plakogiannis, Whitestone, NY (US); Tamanna Lather, Jersey City, NJ (US); Nisarg Modi, Jersey City, NJ (US); Marina Borovinskaya, East Brunswick, NJ (US)

(21) Appl. No.: 17/540,475

(22) Filed: Dec. 2, 2021

Related U.S. Application Data
(60) Provisional application No. 63/120,932, filed on Dec. 3, 2020.

(51) Int. Cl. A61K 31/352; A61K 31/05; A61K 9/00

(52) U.S. Cl. CPC A61K 31/352; A61K 9/0019; A61K 31/05

(57) ABSTRACT

The present disclosure relates to methods of treating cancer and, more particularly, an active agent selected from the group consisting of tetrahydrocannabinol (THC), cannabidiol (CBD), or combinations thereof, in a dosage form for transdermal delivery in the treatment of cancer.

This application claims benefit of Ser. No. 63/120,932 filed Dec. 3, 2020, the entirety of which is incorporated herein by reference.

### BACKGROUND OF THE INVENTION

The present invention, in some embodiments thereof, relates to methods of treating cancer and, more particularly, an active agent selected from the group consisting of tetrahydrocannabinol (THC), cannabidiol (CBD), or combinations thereof, in a dosage form for transdermal delivery in the treatment of cancer.

Cancer is the second leading cause of death in the U.S.A. The estimates for 2014 are that approximately 585,000 people will die of cancer and 1.6 million new cases will be diagnosed (American Cancer Society, Cancer Facts & Figures 2014). Glioblastoma multiforme (GBM) is the most common and most deadly primary brain tumor affecting adults. Despite advancements made in surgical, radiological, and chemo-therapies for this grade IV astrocytoma, prognoses have remained very poor: median survival time from diagnosis remains at 9-15 months with less than 10% of patients surviving beyond 5 years. In the past 10 years, survival in the treatment of this disease has not advanced significantly, with the postoperative standard being the administration of chemoradiotherapy with temozolomide, followed by 6 cycles of sequential chemotherapy with temozolomide. Δ9-tetrahydrocannabinol (THC) and cannabidiol (CBD) have shown a clear synergistic antitumour effect with temozolomide and radiotherapy in preclinical glioma models. THC and CBD have a wide variety of biological effects by binding with and activating the type 1 and type 2 cannabinoid receptors (CBT expressed in certain neuronal areas of the brain and CB2 expressed in the immune system and in glial cells). The activation of these receptors initiates a signaling pathway, called the endoplasmic reticulum stress response, which generates tumour cell autophagy by activating TRB3.

Cannabis (marijuana) is a schedule-I drug in USA. Cannabis is a flowering plant which contains more than 400 phytonutrient (micronutrient). More than 100 different types of terpenoids, essential oils, antioxidants and cannabinoids have been extracted from the plant. From all of the phytochemicals, only tetrahydrocannabinol (THC) showed significant psychoactive effect. A number of research papers have been published on THC due to its psychoactive and therapeutic effects. Apart from THC, several other constituents have been studied, which also showed some therapeutic effect without psychoactive effect such as cannabidiol (CBD), cannbinol (CBN), cannabichromene (CBC), cannabigerol (CBG), tetrahydrocannbivarin (THCV), delta 9-tetrahydrocannbinol (delta9THC) and many more. It has been showed that cannabis and its derivatives can be used for the treatment of pain, type-2 related metabolic disorder, decrease intraocular pressure, Dravet syndrome, Lennox-Gastaut Syndrome (LGS), epilepsy, nausea, pain and wasting associated with AIDS, arthritis and rheumatism, migraines, muscle spasticity associated with multiple sclerosis and paralysis, alcohol and narcotics withdrawal, stress and depression, asthma, fibromyalgia, inflammatory pain, and pain and/or inflammation associated with chemotherapy, act as an antimicrobial. FDA approved Marinol and Syndros contains delta 9-THC, which currently used in treatment of nausea, vomiting, and anorexia associated with chemotherapy treatments. In clinical studies Sativex (cannabinoid extract oromucosal spray containing THC and CBD) has shown improvements in neuropathic pain and sleep quality. Furthermore, in April 2016 FDA gave orphan drug designation to cannabidiol for the treatment of Tuberous Sclerosis Complex (TSC), Dravet Syndrome and Lennox-Gastaut Syndrome. Cannabidiol is an orally effective treatment for pain and inflammation (Costa, B. The non-psychoactive cannabis constituent cannabidiol is an orally effective therapeutic agent in rat chronic inflammatory and neuropathic pain. European Journal of Pharmacology. Volume 556, Issues 1-3, 5 Feb. 2007, Pages 75-83).

The utility of cannabinoids in the treatment of cancer has been of great interest. Recently, both CB1 and CB2 were found to be expressed in many cancer types, but both receptors were often undetectable at the site of the cancers' origin before neoplastic transformation (Sarfaraz, S.; Adhami, V. M.; Syed, D. N.; Afaq, F.; Mukhtar, H. Cannabinoids for Cancer Treatment: Progress and Promise. Cancer Res. 2008, 68, 339-342). Further evidence for the role of endocannabinoid system in neoplasia came when Wang and colleagues showed that CB1 has a tumor-suppressive function in a genetically modified mouse model of colon cancer. (Wang, D.; Wang, H.; Ning, W.; Backlund, M. G.; Dey, S. K.; Dubois, R. N. Loss of cannabinoid receptor 1 accelerates intestinal tumor growth. Cancer Res. 2008, 68, 6468-6476). CB1 is upregulated in hepatocellular carcinoma and Hodgkin lymphoma, and the extent to which CB1 was overexpressed correlated with disease severity in epithelial ovarian carcinoma. (Mukhopadhyay, B.; Schuebel, K.; Mukhopadhyay, P.; Cinar, R.; Godlewski, G.; Xiong, K.; Mackie, K.; Lizak, M.; Yuan, Q.; Goldman, D.; et al. Cannabinoid receptor 1 promotes hepatocellular carcinoma initiation and progression through multiple mechanisms. Hepatology 2015, 61, 1615-1626). CB2 has also been found to be overexpressed in HER2+ breast cancers and gliomas. (Pérez-Gómez, E.; Andradas, C.; Blasco-Benito, S.; Ca arel, M. M.; Garcia-Taboada, E.; Villa-Morales, M.; Moreno, E.; Hamann, S.; Martin-Villar, E.; Flores, J. M.; et al. Role of Cannabinoid Receptor CB2 in HER2 Pro-oncogenic Signaling in Breast Cancer. J. Natl. Cancer Inst. 2015, 107, djv077). It was shown that overexpression of both CB1 and CB2 was correlated with poor prognosis in stage IV colorectal carcinoma. (Jung, C. K.; Kang, W. K.; Park, J. M.; Ahn, H. J.; Kim, S. W.; Oh, S. T.; Choi, K. Y. Expression of the cannabinoid type I receptor and prognosis following surgery in colorectal cancer. Oncol. Lett. 2012, 5, 870-876). The administration of cannabinoids, such as D8-THC, D9-THC, and CBD, inhibited the DNA synthesis and growth of lung adenocarcinoma in cultured cells as well as mouse tumor models. (Carchman, R.; Harris, L. S.; E Munson, A. The inhibition of DNA synthesis by cannabinoids. Cancer Res. 1976, 36, 95-100). Similar effects were seen in both in vitro and in vivo models of various other cancers, including glioma, breast, pancreas, prostate, colorectal carcinoma, and lymphoma. (Bifulco, M.; Laezza, C.; Pisanti, S.; Gazzerro, P. Cannabinoids and cancer: Pros and cons of an antitumour strategy. Br. J. Pharmacol. 2006, 148, 123-135). There are various proposed mechanisms of action behind these findings, including, but not limited to: cell cycle arrest, induction of apoptosis, as well as inhibition of neovascularization, migration, adhesion, invasion, and metastasis. (Ramer, R.; Hinz, B. Cannabinoids as Anticancer Drugs. Cannabinoid Pharmacol. 2017, 80, 397-436). See Seltzer, E S et al. Cannabidiol (CBD) as a Promising Anti-Cancer Drug. Cancers 2020, 12, 3203; doi:10.3390/cancers1213203.

There is extensive preclinical research indicating CBD as an efficacious anti-cancer agent either alone or in conjunction with other cannabinoids, chemotherapies, and/or radiation therapy. There is a need for an improved drug delivery system of CBD and/or THC which can overcome the drawbacks associated with oral routes. Transdermal and/or topical delivery of CBD and/or THC, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorphs thereof, stereoisomers thereof, coated form thereof, ion-pairs thereof, solution thereof in solvents alone or in combinations thereof can address the challenges associated with oral drug delivery, and are useful as treatment, prevention and/or control of, for example, cancer.

All references cited herein are incorporated herein by reference in their entireties.

### BRIEF SUMMARY OF THE INVENTION

The disclosure provides compositions and methods for the treatment and/or prevention and/or control of cancer, for example glioblastoma, GBM, using transdermal drug delivery. In transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week.

Through transdermal delivery, such as transdermal compositions or transdermal formulations or transdermal patch of highly purified tetrahydrocannabinol (THC), cannabidiol (CBD), or combinations thereof, can be applied topically to skin thereby delivering the drug throughout the duration of topical application. Depending on the requirement, the duration of topical application can be once in a day, once in two days, once in three days, once in four days, once in five days, once in a week. Therefore, transdermal delivery can overcome the multiple dose regimen of oral delivery by reducing the dosing frequency.

Moreover, in transdermal drug delivery the drug is delivered slowly and continuously throughout the duration of topical application hence there are no peaks and troughs in drug plasma concentration which are associated with multiple dose administration in a day. Therefore, by transdermal delivery of highly purified tetrahydrocannabinol (THC), cannabidiol (CBD), or combinations thereof, patients can have the therapeutic effect of the drug for extended period of time without drastic changes in drug plasma concentration.

In transdermal delivery drug is delivered into systemic circulation through the skin, it escapes the first pass hepatic metabolism therefore to achieve the desired therapeutic activity less drug is required, resulting into less adverse effects or side effects. Tetrahydrocannabinol (THC) and Cannabidiol (CBD) have high lipid solubility and after oral administration undergoes hepatic first pass metabolism, therefore of the administered dose only 10%-20% reaches systemic circulation, thus as compared to oral dose, transdermal delivery a small dose of tetrahydrocannabinol (THC), cannabidiol (CBD), or combinations thereof can give the desired therapeutic effects at a lower dose than oral.

Furthermore, transdermal delivery is easy, noninvasive and convenient. Administration of a transdermal patch or transdermal composition does not require medical supervision as patients can topically apply the transdermal patch or transdermal composition themselves.

With respect to tetrahydrocannabinol (THC), cannabidiol (CBD), or combinations thereof it is expected that interpatient variability in pharmacologic response will be less with transdermal delivery as drug plasma concentration can be controlled by controlling the rate of drug delivery from transdermal composition or transdermal patch. With transdermal delivery a small amount of tetrahydrocannabinol (THC), cannabidiol (CBD), or combinations thereof can be delivered for longer duration than oral administration. Transdermal formulations of tetrahydrocannabinol (THC), cannabidiol (CBD), or combinations thereof can also provide more abuse deterrence than immediate release dosage forms.

Moreover, in case of any adverse effect, side effect or emergency transdermal delivery gives the liberty to terminate the therapy anytime by taking off the transdermal patch or transdermal composition from skin.

As per above stated reasons for the treatment and/or prevention and/or control of cancer, transdermal delivery can provide patient friendly, simplified and convenient therapeutic regimen over traditional delivery systems. Transdermal delivery can reduce the dosing frequency of highly purified tetrahydrocannabinol (THC), cannabidiol (CBD), or combinations thereof. Depending on the necessity, dosing frequency can be once in a day, once in two days, once in three days, once in four days, once in five days, once in six days, once in a week.

Through transdermal administration of a drug combination, two or more drugs can be delivered simultaneously. Depending on the necessity, dosing frequency of transdermal patch or transdermal composition containing drug combination can be once in a day, once in two days, once in three days, once in four days, once in five days, once in six days, once in a week. It would be a great addition to the patient compliance.

The continuous drug delivery methods and systems as disclosed herein may provide active agent in several different ways, such as through infusion therapy via, for example, intra or subcutaneous application, or transdermal or topical application. Infusion therapy administers medication through the use of a sterile thin tube such as catheter that is inserted into the body and secured. A pump delivery system and any other delivery system that would deliver a continuous infusion such as depot injection and ambulatory pumps can be used for this purpose. Continuous drug delivery systems and methods also may include, for example, transdermal systems. In Transdermal drug delivery, a transdermal patch or transdermal composition is applied topically to the skin surface. Throughout the duration of topical application of a transdermal patch or transdermal composition drug is continuously released and delivered through the intact skin (via transcellular, intercellular and transappendageal routes) to achieve systemic effect. Therefore, once applied transdermal composition or transdermal patch can deliver drug into systemic circulation throughout the day or even for more than one day depending on the duration of its application which can be even up to a week.

In continuous drug delivery, active agent is continuously released and delivered to the patient to achieve systemic effect. Therefore, once applied a continuous drug delivery system can deliver active agents into systemic circulation throughout a day or even for more than one day depending on the duration of its application which can be even up to a week.

Through continuous drug delivery, for example, purified tetrahydrocannabinol (THC), cannabidiol (CBD), or combinations thereof, can be delivered throughout the duration of application. Depending on the requirement, the duration of application can be once in a day, once in two days, once in three days, once in four days, once in five days, once in a week. Therefore, continuous drug delivery can overcome the multiple dose regimen of oral delivery by reducing the dosing frequency.

In continuous drug delivery the pharmaceutical composition comprising active agent is delivered slowly and continuously throughout the duration of administration, hence there are no peaks and troughs in drug plasma concentration of the active agent which are associated with multiple dose administration, such as oral administration, in a day. Therefore, by continuous drug delivery of, for example, tetrahydrocannabinol (THC), cannabidiol (CBD), or combinations thereof, patients can have the therapeutic effect of the drug for extended period of time without drastic changes in drug plasma concentration.

In continuous drug delivery active using the systems and methods as disclosed herein active agent is delivered into systemic circulation and escapes the first pass hepatic metabolism, therefore to achieve the desired therapeutic activity less drug is required, resulting into less adverse effects or side effects. Tetrahydrocannabinol (THC) and Cannabidiol (CBD) have high lipid solubility and after oral administration undergoes hepatic first pass metabolism, therefore of the administered dose only 10%-20% reaches systemic circulation, thus as compared to oral dose, a continuous drug delivery a small dose of tetrahydrocannabinol (THC), cannabidiol (CBD), or combinations thereof can give the desired therapeutic effects at a lower dose than oral.

Furthermore, using a continuous drug delivery system and methods as disclosed herein is easy, noninvasive and convenient. Administration of a drug compound by continuous drug does not require medical supervision as patients can control the administration themselves.

In certain embodiments, the continuous drug delivery system and methods as disclosed herein may comprise a pump device, such as ones which are commonly used to deliver one or more fluids to a targeted individual. For example, a medical infusion pump device may be used to deliver a medicine to a patient as part of a medical treatment. The active agent(s) that is delivered by the infusion pump device can depend on the condition of the patient and the desired treatment plan. For example, infusion pump devices have been used to deliver insulin into the subcutaneous tissue and to the vasculature of diabetes patients to regulate blood-glucose levels. In some circumstances, the dosage of medicine delivered by the infusion pump can be calculated by the infusion pump system. In these circumstances, the infusion pump system can take into account many variables, including user input, when making such calculations.

Other forms of continuous drug delivery systems and methods may make use of fluid delivery to a targeted individual. For example, insulin, glucagon, or another medicine can be injected using a manual syringe or a single use injection “pen.” In some circumstances, an injectable form of glucagon is used in emergency aid of severe hypoglycemia when the victim is unconscious or for other reasons cannot take glucose orally. The glucagon fluid can be rapidly injected to the patient by intramuscular, intravenous, or subcutaneous injection, and quickly raises the blood glucose level of the patient.

One category of devices for delivering such fluids is that of pumps that have been developed for the administration of insulin and other medicaments for those suffering from both type I and type II diabetes. Some pumps configured as portable infusion devices can provide Continuous drug for the treatment of diabetes. Such therapy may include, e.g., the regular and/or continuous injection or infusion of insulin into the skin of a person suffering from diabetes and offer an alternative to multiple daily injections of insulin by an insulin syringe or an insulin pen. Such pumps can be ambulatory/portable infusion pumps that are worn by the user and may use replaceable cartridges. Examples of such pumps and various features that can be associated with such pumps include those disclosed in U.S. patent application Ser. No. 13/557,163, U.S. patent application Ser. No. 12/714,299, U.S. patent application Ser. No. 12/538,018, U.S. patent application Ser. No. 13/838,617, U.S. patent application Ser. No. 13/827,707 and U.S. Pat. No. 8,287,495, each of which is hereby incorporated herein by reference in its entirety.

In certain embodiments as disclosed herein continuous drug delivery system may comprise, for example, a patch pump, or micro pump. Patch pumps are small pumps, typically ambulatory, that are carried directly on the skin under the user's clothing. Such a pump generally is situated directly on the injection site such that no tubing is required to deliver the insulin or other medicament to the patient. Patch pumps typically are at least in part disposable, meant to be worn for a day or two and then discarded for a new patch pump.

The disclosure provides a pharmaceutical composition comprising an active agent selected from the group consisting of tetrahydrocannabinol (THC), cannabidiol (CBD), and combinations thereof, in a dosage form for transdermal delivery. The disclosure provides a pharmaceutical composition comprising an active agent selected from the group consisting of tetrahydrocannabinol (THC), cannabidiol (CBD), and combinations thereof, in a dosage form for topical delivery. The disclosure provides a pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorphs thereof, solid solutions thereof, coated forms thereof, stereoisomers thereof, ion-pairs thereof, solutions thereof, powder forms thereof, liquid forms thereof, alone or combinations thereof. The disclosure provides a pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, active metabolites thereof, polymorphs thereof, solid solutions thereof, coated forms thereof, ion-pairs thereof, stereoisomers thereof, solutions thereof, powder forms thereof, liquid forms thereof, alone or combinations thereof. The disclosure provides a pharmaceutical composition which comprises at least about 0.10% to about 80% (w/w) of the active agent CBD. The disclosure provides a pharmaceutical composition which comprises at least about 0.1% to about 80% of the active agent THC. The disclosure provides a pharmaceutical composition formulated as a transdermal liquid formulation, a transdermal semisolid formulation, or a transdermal polymer matrix formulation. The disclosure provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof. The disclosure provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.10%-99.5% w/w or w/v. The disclosure provides a pharmaceutical composition wherein the carrier is present in the range of 70%-99% w/w or w/v. The disclosure provides a pharmaceutical composition which is a topical pharmaceutical composition which is formulated as metered dose gel, metered dose spray, gel, cream, solution, emulsion, liquid compositions, semisolid compositions, film forming formulations. The disclosure provides a pharmaceutical composition which is formulated as a transdermal patch. The disclosure provides a pharmaceutical composition formulated as a transdermal patch, wherein the transdermal patch is selected from the group such as to reservoir patch, a microreservoir patch, a matrix patch, a pressure sensitive adhesive patch, extended release transdermal film a liquid reservoir system, a microreservoir patch, a matrix patch, a pressure sensitive adhesive patch, a mucoadhesive patch, and combinations thereof. The disclosure provides a pharmaceutical composition indicated for the treatment and/or prevention and/or control of cancer in a patient, wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and ahematological cancer, such as multiple myeloma, B-cell lymphoma, T-cell lymphoma, Hodgkin's lymphoma/primary mediastinal B-cell lymphoma, and chronic myelogenous leukemia. The disclosure provides a pharmaceutical composition which is formulated as the transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, once in 15 days to about 30 days. The disclosure provides a pharmaceutical composition which is formulated as the topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, once in 15 days to about 30 days. The disclosure provides a pharmaceutical composition which may be formulated as microneedles. The disclosure provides a pharmaceutical composition wherein said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route. The disclosure provides a pharmaceutical composition wherein said tetrahydrocannabinol (THC), cannabidiol (CBD), or derivative thereof is produced by a synthetic route. The disclosure provides a pharmaceutical composition co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

The disclosure provides a method for the treatment and/or prevention and/or control of cancer in a patient comprising: selecting a patient in need of treatment and/or prevention and/or control of cancer; topically applying the transdermal pharmaceutical composition as disclosed herein. The disclosure provides a method wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, and chronic myelogenous leukemia. The disclosure provides a method wherein the cancer is glioblastoma, GBM. The disclosure provides a method wherein the topical application of a transdermal pharmaceutical composition for the treatment and/or prevention and/or control of cancer in a patient, wherein the transdermal pharmaceutical composition is applied at a time period selected from the group consisting of once in a day, once in two days, once in three days, once in four days, once in five days, once in six days, once in a week, and once in ten days. The disclosure provides a method further providing a constant rate of delivery of the active components of the transdermal pharmaceutical composition over a time period. The disclosure provides a method further providing a steady absorption rates of the active components of the transdermal pharmaceutical composition over a time period. The disclosure provides a method further achieving a constant blood serum levels of the active components of the transdermal pharmaceutical composition over a time period. The disclosure provides a method further achieving a reduced variability in dosage of the active components of the transdermal pharmaceutical composition over a time period. The disclosure provides a method further providing a plasma concentration of the active components of the transdermal pharmaceutical composition in a therapeutic range over a period of time. The disclosure provides a method further providing a plasma concentration of the active components of the transdermal pharmaceutical composition in a therapeutic range of about 0.1 ng/mL to about 500 ng/mL. The disclosure provides a method wherein the transdermal pharmaceutical composition is in a dosage form selected from the group consisting of: cream, lotion, gel, oil, ointment, suppository, spray, foam, liniment, aerosol, buccal and sublingual tablet, a transdermal device, and a transdermal patch. The disclosure provides a method wherein the pharmaceutical composition is co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

The disclosure provides a pharmaceutical composition comprising at least one active agent selected from the group consisting of: about 3% to about 15% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof, about 3% to about 15% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof, and combinations thereof, further wherein the pharmaceutical composition comprises: about 0% to about 97% of at least one solvent; about 0% to about 97% of at least surfactant; optionally, about 0% to about 97% of at least one permeation enhancer; and/or optionally, about 0% to about 97% of an adhesive and/or polymer. The disclosure provides a pharmaceutical composition wherein the active agent is present at a concentration selected from the group consisting of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, and about 50% of the formulation. The disclosure provides a pharmaceutical composition wherein the active agent is present at a concentration selected from the group consisting of about 1% to about 10%, about 1% to about 9%, about 1% to about 8%, about 1% to about 7%, about 1% to about 6%, about 10% to about 5%, about 0.1 to about 50%, about 1 to 20%, of about 5% to 25%, about 10% to about 20%, or about 15% to about 18%, about 30% to about 70%, and about 35% to about 65% of the formulation. The disclosure provides a pharmaceutical composition wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof. The disclosure provides a pharmaceutical composition wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof. The disclosure provides a pharmaceutical composition wherein the pharmaceutical composition comprises one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery.

The disclosure provides a pharmaceutical composition wherein the pharmaceutical composition is formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, a formulation in an infusion pump, or transdermal drug-in-adhesive matrix formulation. The disclosure provides a pharmaceutical composition wherein the pharmaceutical composition provides a continuous, sustained delivery of the pharmaceutical composition to mitigate peak and valley pharmacokinetic behavior of the active agent. The disclosure provides a pharmaceutical composition wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof. The disclosure provides a pharmaceutical composition wherein the pharmaceutical composition provides a continuous, sustained delivery of the pharmaceutical composition via intravenous or subcutaneous infusion. The disclosure provides a pharmaceutical composition wherein the pharmaceutical composition provides a continuous, sustained delivery of the pharmaceutical composition via subcutaneous infusion. The disclosure provides a pharmaceutical composition wherein the pharmaceutical composition provides a continuous, sustained delivery of the pharmaceutical composition via an infusion pump device. The disclosure provides a pharmaceutical composition wherein the pharmaceutical composition provides a continuous, sustained delivery of the pharmaceutical composition via an ambulatory/portable infusion pump that is worn by the patient and may use replaceable cartridges. The disclosure provides a pharmaceutical composition wherein the pharmaceutical composition provides a continuous, sustained delivery of the pharmaceutical composition via a patch pump or micro pump. The disclosure provides a pharmaceutical composition wherein the pharmaceutical composition is formulated as a topical liquid formulation, topical semisolid formulation, topical gel formulation, topical polymer matrix formulation, topical adhesive matrix formulation, topical film forming gel formulation, a formulation in an infusion pump, or topical film forming spray formulation. The disclosure provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof. The disclosure provides a pharmaceutical composition further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.10%-99.5% w/w or w/v. The disclosure provides a pharmaceutical composition wherein the pharmaceutical composition is formulated as a transdermal patch. The disclosure provides a pharmaceutical composition wherein the pharmaceutical composition is formulated as a multilayer transdermal matrix system, a metered dose transdermal gel, metered dose transdermal spray, a film forming gel, a film forming spray, or a meter-dose aerosol. The disclosure provides a pharmaceutical composition wherein the pharmaceutical composition is formulated as a topical patch. The disclosure provides a pharmaceutical composition wherein the pharmaceutical composition is formulated as metered dose gel, metered dose spray, gel, cream, solution, emulsion, liquid compositions, semisolid compositions, a matrix of adhesive in combination with polymers, or film forming formulations. The disclosure provides a pharmaceutical composition wherein the pharmaceutical composition is formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof. The disclosure provides a pharmaceutical composition wherein the pharmaceutical composition is formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof. The disclosure provides a pharmaceutical composition wherein the pharmaceutical composition is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a pharmaceutical composition wherein the pharmaceutical composition is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days. The disclosure provides a pharmaceutical composition wherein the pharmaceutical composition is formulated as microneedles. The disclosure provides a pharmaceutical composition wherein the said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route. The disclosure provides a pharmaceutical composition indicated for the treatment and/or prevention and/or control of cancer in a patient, wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, and chronic myelogenous leukemia. The disclosure provides a pharmaceutical composition wherein said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route. The disclosure provides a pharmaceutical composition wherein said tetrahydrocannabinol (THC), cannabidiol (CBD), or derivative thereof is produced by a synthetic route. The disclosure provides a pharmaceutical composition wherein the active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition. The disclosure provides a pharmaceutical composition wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, and about 5 μg/mL. The disclosure provides a pharmaceutical composition co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

The disclosure provides a method for the treatment and/or prevention and/or control of cancer in a patient comprising: selecting a patient in need of treatment and/or prevention and/or control of cancer; topically applying the pharmaceutical composition as disclosed herein, thereby treating and/or preventing and/or controlling cancer in the patient. The disclosure provides a method for the treatment and/or prevention and/or control of cancer in a patient wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, and chronic myelogenous leukemia. The disclosure provides a method for the treatment and/or prevention and/or control of cancer in a patient wherein the cancer is glioblastoma, GBM. The disclosure provides a method for the treatment and/or prevention and/or control of cancer in a patient wherein the topical application of a transdermal pharmaceutical composition for the treatment and/or prevention and/or control of cancer in a patient, wherein the transdermal pharmaceutical composition is applied at a time period selected from the group consisting of once in a day, once in two days, once in three days, once in four days, once in five days, once in six days, once in a week, and once in ten days. The disclosure provides a method for the treatment and/or prevention and/or control of cancer in a patient further providing a constant rate of delivery of the active components of the transdermal pharmaceutical composition over a time period. The disclosure provides a method for the treatment and/or prevention and/or control of cancer in a patient further providing a steady absorption rates of the active components of the transdermal pharmaceutical composition over a time period. The disclosure provides a method for the treatment and/or prevention and/or control of cancer in a patient further achieving a constant blood serum levels of the active components of the transdermal pharmaceutical composition over a time period. The disclosure provides a method for the treatment and/or prevention and/or control of cancer in a patient further achieving a reduced variability in dosage of the active components of the transdermal pharmaceutical composition over a time period. The disclosure provides a method for the treatment and/or prevention and/or control of cancer in a patient further providing a plasma concentration of the active components of the transdermal pharmaceutical composition in a therapeutic range over a period of time. The disclosure provides a method for the treatment and/or prevention and/or control of cancer in a patient further providing a plasma concentration of the active components of the transdermal pharmaceutical composition in a therapeutic range of about 0.1 ng/mL to about 500 ng/mL. The disclosure provides a method for the treatment and/or prevention and/or control of cancer in a patient wherein the continuous delivery system provides a continuous, sustained delivery of the pharmaceutical composition to the patient via intravenous or subcutaneous infusion. The disclosure provides a method for the treatment and/or prevention and/or control of cancer in a patient wherein the continuous delivery system provides a continuous, sustained delivery of the pharmaceutical composition to the patient via subcutaneous infusion. The disclosure provides a method for the treatment and/or prevention and/or control of cancer in a patient wherein the continuous delivery system provides a continuous, sustained delivery of the pharmaceutical composition to the patient via an infusion pump device. The disclosure provides a method for the treatment and/or prevention and/or control of cancer in a patient wherein the continuous delivery system provides a continuous, sustained delivery of the pharmaceutical composition to the patient via an ambulatory/portable infusion pump that is worn by the patient and may use replaceable cartridges. The disclosure provides a method for the treatment and/or prevention and/or control of cancer in a patient wherein the continuous delivery system provides a continuous, sustained delivery of the pharmaceutical composition to the patient via a patch pump or micro pump. The disclosure provides a method for the treatment and/or prevention and/or control of cancer in a patient wherein the continuous delivery system comprises a pharmaceutical composition selected from the group consisting of a liquid formulation, a solid formulation, a semi-solid formulation, an emulsion formulation, a nanoparticle formulation, a matrix formulation, a film formulation, a patch formulation, a formulation in an infusion pump, and/or combinations thereof. The disclosure provides a method for the treatment and/or prevention and/or control of cancer in a patient wherein the transdermal pharmaceutical composition is in a dosage form selected from the group consisting of: cream, lotion, gel, oil, ointment, suppository, spray, foam, liniment, aerosol, buccal and sublingual tablet, a transdermal device, and a transdermal patch. The disclosure provides a method for the treatment and/or prevention and/or control of cancer in a patient wherein the pharmaceutical composition is co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

The disclosure provides for the use of the compositions of the disclosure for the production of a medicament for preventing and/or treating the indications as set forth herein.

In accordance with a further embodiment, the present disclosure provides a use of the pharmaceutical compositions described above, in an amount effective for use in a medicament, and most preferably for use as a medicament for treating a disease or disorder, for example, as set forth in herein, in a subject.

In accordance with yet another embodiment, the present disclosure provides a use of the pharmaceutical compositions described above, and at least one additional therapeutic agent, in an amount effective for use in a medicament, and most preferably for use as a medicament for treating a disease or disorder associated with disease, for example, as set forth herein, in a subject.

The disclosure provides a method for treating and/or preventing a disease or condition as set forth herein in a patient, wherein said method comprises: selecting a patient in need of treating and/or preventing said disease or condition as set forth herein; administering to the patient a composition of the disclosure in a therapeutically effective amount, thereby treating and/or preventing said disease in said patient.

### DETAILED DESCRIPTION OF THE INVENTION

It is to be understood that this invention is not limited to particular embodiments described, as such may, of course, vary. It is also to be understood that the terminology used herein is for the purpose of describing particular embodiments only, and is not intended to be limiting, since the scope of this invention will be limited only by the appended claims.

The detailed description of the invention is divided into various sections only for the reader's convenience and disclosure found in any section may be combined with that in another section. Unless defined otherwise, all technical and scientific terms used herein have the same meaning as commonly understood by one of ordinary skill in the art to which this invention belongs.

It must be noted that as used herein and in the appended claims, the singular forms “a”, “an”, and “the” include plural referents unless the context clearly dictates otherwise. Thus, for example, reference to “a compound” includes a plurality of compounds.

As used herein, the term “pharmaceutically acceptable salts” includes acid addition salts or addition salts of free bases. The term “pharmaceutically acceptable salts” of the active agent within its scope all the possible isomers and their mixtures, and any pharmaceutically acceptable metabolite, bioprecursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being.

As used herein, the terms “subject” and “patient” are used interchangeably. As used herein, the term “patient” refers to an animal, preferably a mammal such as a non-primate (e.g., cows, pigs, horses, cats, dogs, rats etc.) and a primate (e.g., monkey and human), and most preferably a human. In some embodiments, the subject is a non-human animal such as a farm animal (e.g., a horse, pig, or cow) or a pet (e.g., a dog or cat). In a specific embodiment, the subject is a human. In some embodiments, a subject may be suffering from, and/or susceptible to a disease, disorder, and/or condition (e.g., cancer). In some embodiments, a patient is a human that has been diagnosed with a cancer. In some embodiments, a patient is a human possessing one or more female reproductive organs. In some embodiments, a patient is a human female (e.g., a woman) that has been diagnosed with a gynecological cancer or breast cancer (e.g., a cancer such as ovarian cancer, cancer of the fallopian tube(s), peritoneal cancer and breast cancer). As used herein, a “patient population” or “population of subjects” refers to a plurality of patients or subjects.

As used herein, the term “agent” refers to any molecule, compound, methodology and/or substance for use in the prevention, treatment, management and/or diagnosis of a disease or condition. As used herein, the term “effective amount” refers to the amount of a therapy that is sufficient to result in the prevention of the development, recurrence, or onset of a disease or condition, and one or more symptoms thereof, to enhance or improve the prophylactic effect(s) of another therapy, reduce the severity, the duration of a disease or condition, ameliorate one or more symptoms of a disease or condition, prevent the advancement of a disease or condition, cause regression of a disease or condition, and/or enhance or improve the therapeutic effect(s) of another therapy.

As used herein, the phrase “pharmaceutically acceptable” means approved by a regulatory agency of the federal or a state government, or listed in the U.S. Pharmacopeia, European Pharmacopeia, or other generally recognized pharmacopeia for use in animals, and more particularly, in humans.

As used herein, the term “therapeutic agent” refers to any molecule, compound, and/or substance that is used for treating and/or managing a disease or disorder.

As used herein, the terms “therapies” and “therapy” can refer to any method(s), composition(s), and/or agent(s) that can be used in the prevention, treatment and/or management of a disease or condition, or one or more symptoms thereof. In certain embodiments, the terms “therapy” and “therapies” refer to small molecule therapy.

The term “derivative” or “derivatized” as used herein includes, for example, chemical modification of a compound of the disclosure, or extracted from botanical sources or pharmaceutically acceptable salts thereof or mixtures thereof. That is, a “derivative” may be a functional equivalent of a compound of the disclosure, which is capable of inducing the improved pharmacological functional activity in a given subject.

As used herein, the terms “composition” and “formulation” are used interchangeably.

As used herein, the term “transdermal delivery” means delivery of drug into systemic circulation through the skin.

Unless defined otherwise, all technical and scientific terms used herein have the same meaning as commonly understood by one of ordinary skill in the art to which this invention belongs. As used herein the following terms have the following meanings.

### Active Agent

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. Disclosure provides for, for example, transdermal formulations comprising one or more of the following active agents, for example, tetrahydrocannabinol (THC), cannabidiol (CBD), and combinations thereof.

The term “active ingredient” refers to an agent, active ingredient compound or other substance, or compositions and mixture thereof that provide some pharmacological, often beneficial, effect. Reference to a specific active ingredient shall include where appropriate the active ingredient and it's pharmaceutically acceptable salts. The disclosure provides for, for example, transdermal formulations and/or topical formulations comprising one or more of the following active agents: Cannabinoids are a group of 21-carbon-containing terpenophenolic compounds produced by Cannabis species. Cannabinoids may also be synthetically produced. The term “cannabinoid” refers hereinafter to a class of diverse chemical compounds that act on cannabinoid receptors on cells that repress neurotransmitter release in the brain. These receptor proteins include the endocannabinoids (produced naturally in the body by humans and animals), the phytocannabinoids (found in cannabis and some other plants), and synthetic cannabinoids. Lipophilic cannabinoids are generally grouped as endocannabinoids (most typically as mammalian endocannabinoids); phytocannabinoids, from plant sources; and synthetic cannabinoids. Such cannabinoids are also often classified into the following subclasses: Cannabigerols (CBG); Cannabichromenes (CBC); Cannabidiol (CBD; CBDL); Tetrahydrocannabinol (THC); Cannabinol (CBN); Cannabicyclol (CBL); Cannabielsoin (CBE); and, Cannabitriol (CBT).

Cannabidiol IUPAC Name 2-[(1R,6R)-6-isopropenyl-3-methylcyclohex-2-en-1-yl]-5-pentylbenzene-1,3-diol Chemical Formula: C21H3002 Molecular weight: 314.46 dalton Chemical structure is shown below as formula I

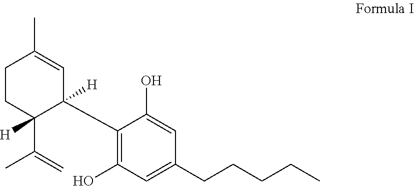 [SMILES: C=C(C)[C@@H]1CCC(C)=C[C@H]1c1c(O)cc(CCCCC)cc1O]

Tetrahydrocannbinol (THC) IUPAC Name (−)-(6aR,10aR)-6,6,9-Trimethyl-3-pentyl-6a,7,8,10a-tetrahydro-6H-benzo[c]chromen-1-ol Chemical Formula: C₂₁H₃₀O₂ Molecular weight: 314.47 dalton.
Chemical structure is shown below as formula II

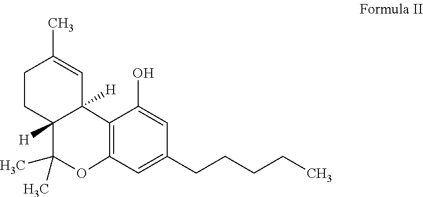 [SMILES: CCCCCc1cc(O)c2c(c1)OC(C)(C)[C@@H]1CCC(C)=C[C@@H]21]

As used herein, the word cannabis refers to all pharmaceutically acceptable forms of cannabis and its derivatives either alone or in combinations thereof, for example, in following forms but not limited to such as free base or salts or isomers or amorphous or crystalline or co crystalline or solid solution or prodrugs or analogs or derivatives or metabolites or polymorphs or its stereoisomer or coated form or ion-pairs. For example, cannabidiol's free base or its salts or its isomers or its amorphous form or its crystalline form or its co crystalline form or its solid solution or its prodrugs or its analogs or its derivatives or synthetic forms or its polymorphs or its stereoisomer or its ion-pairs. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts.

As used herein, the term “cannabidiol” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, polymorph thereof, stereoisomers thereof, powder form thereof, liquid form thereof, ion-pairs thereof, solution of cannabidiol in solvents such as but not limited to methanol, etc. alone or in combinations thereof.

As used herein, the term “THC” includes the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, solid solution thereof, powder form thereof, liquid form thereof, ion-pairs thereof, polymorph thereof, stereoisomers thereof, solution of THC in solvents such as but not limited to methanol, heptane, etc. alone or in combinations thereof.

As used herein, synthetic cannabinoids include at least the following: AM-087 is an analgesic drug that is a cannabinoid agonist derivative of Δ8THC substituted on the 3-position side chain and a potent CB1 agonist; AM-251 is an inverse agonist at the CB1 cannabinoid receptor with close structural similarity to SR141716A (rimonabant), both of which are biarylpyrazole cannabinoid receptor antagonists as well as μ-opioid receptor antagonist; Methanandamide (AM-356) is a stable chiral analog of anandamide and acts on the cannabinoid receptors with a Ki of 17.9 nM at CB1 and 868 nM at CB2; AM-374-palmitylsulfonyl fluoride; AM-381-stearylsulfonyl fluoride; AM404, also known as N-arachidonoylaminophenol, is an active metabolite of paracetamol (acetaminophen) thought to induce its analgesic action through its activity on the endocannabinoid, COX, and TRPV systems, all of which are present in pain and thermoregulatory pathways; AM-411 is an analgesic that is a cannabinoid agonist; AM-411 is a potent and fairly selective CB1 full agonist and produces similar effects to other cannabinoid agonists such as analgesia, sedation, and anxiolysis; AM-630 (6-lodopravadoline) acts as a potent and selective inverse agonist for the cannabinoid receptor CB2, selectivity over CB1 where it acts as a weak partial agonist; AM-661—1-(N-methyl-2-piperidine)methyl-2-methyl-3-(2-iodo)benzoylindole; JWH-018 (1-pentyl-3-(1-naphthoyl)indole) or AM-678 is an analgesic chemical from the naphthoylindole family that acts as a full agonist at both the CB1 and CB2 cannabinoid receptors, with some selectivity for CB2; AM-679 acts as a moderately potent agonist for the cannabinoid receptors; AM-694 (1-(5-fluoropentyl)-3-(2-iodobenzoyl)indole) acts as a potent and selective agonist for the cannabinoid receptor CB1; AM-735—3-bornyl-Δ8-THC, a mixed CB1/CB2 agonist; AM-855 is an analgesic cannabinoid agonist at both CB1 and CB2 with moderate selectivity for CB1; AM-881—a chlorine-substituted stereoisomer of anandamide whose Ki=5.3 nM at CB1 and 95 nM at CB2; AM-883 an allyl-substituted stereoisomer of anandamide whose Ki=9.9 nM at CB1 and 226 nM at CB2; AM-905 is an analgesic cannabinoid which acts as a potent and reasonably selective agonist for the CB1 cannabinoid receptor; AM-906 is an analgesic drug which is a cannabinoid agonist and is a potent and selective agonist for the CB1 cannabinoid receptor; AM-919 is an analgesic cannabinoid receptor agonist, potent with respect to both CB1 and CB2; AM-926—a potent agonist at both CB1 and CB2 with moderate selectivity for CB1; AM-938 is an analgesic drug which is a cannabinoid receptor agonist and while it is still a potent agonist at both CB1 and CB2, it is reasonably selective for CB2; AM-1116—a dimethylated stereoisomer of anandamide; AM-1172—an endocannabinoid analog specifically designed to be a potent and selective inhibitor of AEA uptake that is resistant to FAAH hydrolysis; AM-1220 is a potent and moderately selective agonist for the cannabinoid receptor CB1; AM-1221 acts as a potent and selective agonist for the cannabinoid receptor CB2; AM-1235 (1-(5-fluoropentyl)-3-(naphthalen-1-oyl)-6-nitroindole) acts as a potent and reasonably selective agonist for the cannabinoid receptor CB1; AM-1241 (1-(methylpiperidin-2-ylmethyl)-3-(2-iodo-5-nitrobenzoyl)indole) is a potent and selective agonist for the cannabinoid receptor CB2, with analgesic effects in mammals, particularly against “atypical” pain such as hyperalgesia and allodynia, and has also shown efficacy in the treatment of amyotrophic lateral sclerosis in mammalian models; AM-1248 acts as a moderately potent agonist for both the cannabinoid receptors CB1 and CB2; AM-1710—a CB2 selective cannabilactone with 54× selectivity over CB1; AM-1714 acts as a reasonably selective agonist of the peripheral cannabinoid receptor CB2 and has both analgesic and anti-allodynia effects; AM-2201 (1-(5-fluoropentyl)-3-(1-naphthoyl)indole) acts as a potent but nonselective full agonist for the cannabinoid receptor; AM-2212—a potent agonist at both CB1 and CB2; AM-2213—a potent agonist at both CB1 and CB2; AM-2232 (1-(4-cyanobutyl)-3-(naphthalen-1-oyl)indole) acts as a potent but unselective agonist for the cannabinoid receptors CB1 and CB2; AM-2233 acts as a highly potent full agonist for the cannabinoid receptors CB1 and CB2 and has been found to fully substitute for THC in certain mammalian studies, with a potency lower than that of JWH-018 but higher than WIN 55,212-2; AM-2389 acts as a potent and reasonably selective agonist for the CB1 receptor; AM-3102—an analog of oleoylethanolamide, (the endogenous agonist for proliferator-activated receptor α (PPARα)) it acts as a weak cannabinoid agonist at CB1 and at CB2; AM-4030 an analgesic which is potent agonist at both CB1 and CB2, but also reasonably selective for CB1; AM-4054 is a potent but slow-onset agonist with CB1 affinity and selectivity CB1 over CB2; AM-4113—a CB1 selective neutral antagonist; AM-6545 acts as a peripherally selective silent antagonist for the CB1 and was developed for the treatment of obesity; JWH-007—an analgesic which acts as a cannabinoid agonist at both the CB1 receptor and CB2 receptors, with some selectivity for CB2, JWH-007 is an analgesic which acts as a cannabinoid agonist at both the CB1 and CB2 receptors; JWH-015 acts as a subtype-selective cannabinoid agonist which binds almost 28× more strongly to CB2 than CB1. and has been shown to have immunomodulatory effects, and may be useful in the treatment and/or prevention and/or control of cancer, such as glioblastoma, GBM, JWH-018 an analgesic which acts as a full agonist at both the CB1 and CB2 cannabinoid receptors and produces effects similar to those of THC; JWH-019—an agonist at both CB1 and CB2 receptors and is an analgesic from the naphthoylindole family that acts as a cannabinoid agonist at both the CB1 and CB2 receptors; JWH-030—an analgesic which is a partial agonist at CB1 receptors; JWH-047—a potent and selective agonist for the CB2 receptor, JWH-048—a potent and selective agonist for the CB2 receptor, JWH-051—an analgesic with a high affinity for the CB1 receptor, but is a much stronger agonist for CB2, JWH-057—a 1-deoxy analog of Δ8-THC that has very high affinity for the CB2 receptor, but also has high affinity for the CB1 receptor; JWH-073—an analgesic which acts as a cannabinoid agonist at both the CB1 and CB2 receptors. It is somewhat selective for the CB1 subtype; JWH-081—an analgesic which acts as an agonist at both the cannabinoid CB1 AND CB2 receptors; JWH-098—a potent and fairly selective CB2 agonist; JWH-116—a CB1 ligand; JWH-120—a potent and 173-fold selective CB2 agonist; JWH-122—a potent and fairly selective CB1 agonist; JWH-133—a potent and highly selective CB2 receptor agonist; 1JWH-139—3-(1,1-dimethylpropyl)-6,6,9-trimethyl-6a,7,10,10a-tetrahydro-6H-benzo[c]chromene; JWH-147—an analgesic from the naphthoylpyrrole family, which acts as a cannabinoid agonist at both the CB1 and CB2 receptors; JWH-148—a moderately selective ligand for the CB2 receptor, with more than 8 times selectivity over the CB1 subtype; JWH-149—a potent and fairly selective CB2 agonist; JWH-161—a CB1 ligand; JWH-164—a potent cannabinoid agonist; JWH-166—a potent and highly selective CB2 agonist; JWH-167—a weak cannabinoid agonist from the phenylacetylindole family; JWH-171—an analgesic which acts as a cannabinoid receptor agonist; JWH-175—(1-pentylindol-3-yl)naphthalen-1-ylmethane, 22 nM at CB1, JWH-176—1-([(1E)-3-pentylinden-1-ylidine]methyl)naphthalene; JWH-181—a potent cannabinoid agonist; JWH-182—a potent cannabinoid agonist with some selectivity for CB1; JWH-184—1-pentyl-1H-indol-3-yl-(4-methyl-1-naphthyl)methane; JWH-185—1-pentyl-1H-indol-3-yl-(4-methoxy-1-naphthyl)methane; JWH-192—(1-(2-morpholin-4-ylethyl)indol-3-yl)-4-methylnaphthalen-1-ylmethane; JWH-193—(1-(2-morpholin-4-ylethyl)indol-3-yl)-4-methylnaphthalen-1-ylmethanone; JWH-194—2-methyl-1-pentyl-1H-indol-3-yl-(4-methyl-1-naphthyl)methane; JWH-195—(1-(2-morpholin-4-ylethyl)indol-3-yl)-naphthalen-1-ylmethane; JWH-196—2-methyl-3—(1-naphthalenylmethyl)-1-pentyl-1H-Indole; JWH-197—2-methyl-1-pentyl-1H-indol-3-yl-(4-methoxy-1-naphthyl)methane; JWH-198—(1-(2-morpholin-4-ylethyl)indol-3-yl)-4-methoxynaphthalen-1-ylmethanone; JWH-199—(1-(2-morpholin-4-ylethyl)indol-3-yl)-4-methoxynaphthalen-1-ylmethane; JWH-200—an analgesic from the aminoalkylindole family, which acts as a cannabinoid receptor agonist; JWH-203—an analgesic from the phenylacetylindole family, which acts as a cannabinoid agonist with approximately equal affinity at both the CB1 and CB2 receptors; JWH-205—142-methyl-1-pentylindol-3-yl)-2-phenylethanone; JWH-210—an analgesic chemical from the naphthoylindole family, which acts as a potent cannabinoid agonist at both the CB1 and CB2 receptors; JWH-213—a potent and fairly selective CB2 agonist; JWH-229—1-methoxy-3-(1′,1′-dimethylhexyl)-Δ8-THC, a dibenzopyran cannabinoid which is a potent CB2 agonist; JWH-234—a cannabinoid agonist with selectivity for CB2; JWH-250—an analgesic from the phenylacetylindole family, which acts as a cannabinoid agonist at both the CB1 and CB2 receptors; JWH-251—(1-pentyl-3-(2-methylphenylacetyl)indole); JWH-258—a potent and mildly selective CB1 agonist; JWH-302—(1-pentyl-3-(3-methoxyphenylacetyl)indole); JWH-307—an analgesic from the naphthoylpyrrole family, which acts as a cannabinoid agonist at both the CB1 and CB2 receptors that is somewhat selective for the CB2 subtype; JWH-350—a 11-nor-1-methoxy-3-(1′,1′-dimethylheptyl)-9α-hydroxyhexahydrocannabinol has a 33-fold selectivity for the CB2 receptor and high CB2receptor affinity with little affinity for the CB1 receptor; JWH-359—a dibenzopyran cannabinoid that is a potent and selective CB2 receptor agonist; JWH-387—1-pentyl-3-(4-bromo-1-naphthoyl)indole, an analgesic from the naphthoylindole family, which acts as a potent cannabinoid agonist at both receptors CB1 and CB2; JWH-398—an analgesic chemical from the naphthoylindole family, which acts as a potent cannabinoid agonist at both receptors with a Ki of 2.3 nM at CB1 and 2.8 nM at CB2; JWH-424—a potent and moderately selective CB2 agonist with a Ki of 5.44 nM at CB2 and 20.9 nM at CB1; HU-210 is a cannabinoid that is 100 to 800 times more potent than natural THC from cannabis and has an extended duration of action and is a potent analgesic with many of the same effects as natural THC; Ajulemic acid (AB-III-56, HU-239, IP-751, CPL 7075, CT-3, Resunab) is a cannabinoid derivative of the non-psychoactive THC metabolite 11-nor-9-carboxy-THC that shows useful analgesic and anti-inflammatory effects without causing a subjective “high”. It is being developed for the treatment of neuropathic pain and inflammatory conditions such as arthritis and for the treatment of orphan life-threatening inflammatory diseases; HU-243 (AM-4056) is a cannabinoid which is a potent agonist at both the CB1 and CB2 receptors; HU-308 acts as a cannabinoid agonist and is highly selective for the CB2 receptor subtype. It has analgesic effects, promotes proliferation of neural stem cells, and protects both liver and blood vessel tissues against oxidative stress via inhibition of TNF-α; HU-331 is a quinone anticarcinogenic synthesized from cannabidiol; HU-336 is a strongly antiangiogenic compound, it inhibits angiogenesis by directly inducing apoptosis of vascular endothelial cells without changing the expression of pro- and anti-angiogenic cytokines and their receptors; HU-345 (cannabinol quinone) is a drug that is able to inhibit aortic ring angiogenesis more potently than its parent compound cannabinol; CP 47,497 or (C7)-CP 47,497 is a cannabinoid receptor agonist drug.

The disclosure also provides methods for the biosynthesis of cannabinoids and for the use of a eukaryotic or prokaryotic expression system for the production of biosynthetic enzymes that can be used for the manufacture of cannabinoids and cannabinoid analogs. Yeast as well as eukaryotic and prokaryotic cells are suitable for the cloning and expression of the cannabinoid acid synthase enzymes and include without limitation E. coli, yeast and baculovirus hosts. Thus, the present disclosure provides a method for the production of biosynthetic cannabinoids, such as for example THC and/or CBD, using cannabinoid acid synthase enzymes including, but not limited to, tetrahydrocannabinolic acid (THCA) synthase and cannabidiolic acid (CBDA) synthase. The disclosure further provides for the transdermal and/or topical compositions as disclosed herein comprising, for example, biosynthetic CBD, alone or in combination with other active agents.

According to certain embodiments, transdermal and/or topical compositions described herein are for the prevention and/or control and/or treatment of cancer.

According to certain embodiments described herein, pharmaceutical composition or transdermal formulation or topical formulation contains cannabidiol and/or THC-the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, stereoisomers thereof, coated form thereof, solid solution thereof, ion-pairs thereof, solution thereof in solvents alone or in combinations thereof. More preferably transdermal and topical formulation may include cannabidiol, the free base thereof, salts thereof, isomers thereof, amorphous forms thereof, crystalline forms thereof, co crystalline forms thereof, prodrugs thereof, analogs thereof, derivatives thereof, synthetic forms thereof, biosynthetic forms thereof, active metabolites thereof, polymorphs thereof, ion-pairs thereof, stereoisomers thereof, coated form thereof, solution of cannabidiol in methanol, alone or in combinations thereof. More preferably transdermal and topical formulation may include THC, the free base thereof, salts thereof, isomers thereof, amorphous forms thereof, crystalline forms thereof, co crystalline forms thereof, prodrugs thereof, analogs thereof, derivatives thereof, ion-pairs thereof, synthetic forms thereof, biosynthetic forms thereof, active metabolites thereof, polymorphs thereof, stereoisomers thereof, coated form thereof, solution of cannabidiol in methanol, alone or in combinations thereof.

As used herein, the word active agent refers to all pharmaceutically acceptable forms of the active agent and its derivatives either alone or in combinations thereof, for example, in following forms but not limited to such as free base or salts or isomers or amorphous or crystalline or co crystalline or solid solution or prodrugs or analogs or derivatives or metabolites. For example, the active agent's free base or its salts or its isomers or its amorphous form or its crystalline form or its co crystalline form or its solid solution or its prodrugs or its analogs or its derivatives or synthetic forms. The compound may be in the form of, for example, a pharmaceutically acceptable salt, such as an acid addition salt or a base salt, or a solvate thereof, including a hydrate thereof. Suitable acid addition salts are formed from acids which form non-toxic salts and examples are the hydrochloride, hydrobromide, hydroiodide, sulphate, bisulphate, nitrate, phosphate, hydrogen phosphate, acetate, maleate, fumarate, lactate, tartrate, citrate, gluconate, succinate, saccharate, benzoate, methanesulphonate, ethanesulphonate, benzenesulphonate, p-toluenesulphonate and pamoate salts. Suitable base salts are formed from bases which form non-toxic salts and examples are the sodium, potassium, aluminium, calcium, magnesium, zinc and diethanolamine salts. The active ingredient(s) can be present in the form of a free base or in the form of pharmaceutically acceptable salts. Pharmaceutically acceptable salts forming part of this invention are intended to define but not limited to salts of the carboxylic acid moiety such as alkali metal salts like Li, Na and K salts; alkaline earth metal salts like Ca and Mg salts; salts of organic bases such as lysine, arginine, guanidine, diethanolamine, choline, and the like; ammonium or substituted ammonium salts and aluminium salts. Salts may be acid addition salts which defines but not limited to sulfates, nitrates, phosphates, perchlorates, borates, hydrohalides, acetates, tartrates, maleates, citrates, succinates, palmoates, methanesulfonates, benzoates, salicylates, hydroxynaphthoates, benzensulfonates, ascorbates, glycerophosphates, ketoglutarates and the like.

According to embodiments provided herein, a continuous delivery system comprises a formulation selected from the group consisting of a liquid formulation, a solid formulation, a semi-solid formulation, an emulsion formulation, a nanoparticle formulation, a matrix formulation, a film formulation, a patch formulation, an infusion pump, and/or combinations thereof. The embodiments are intended to be formulated to provide a continuous, sustained delivery to mitigate the peak and valley pharmacokinetic behavior associated with standard immediate release oral delivery forms. The embodiments are intended to be formulated to provide a route of administration selected from the group consisting of an oral, buccal, mucosal, rectal, transdermal, topical, parenteral, and or implantable, and or combinations thereof.

As used herein, the term “active agent” includes the free base thereof, salts thereof, isomers thereof, amorphous forms thereof, crystalline forms thereof, co crystalline forms thereof, prodrugs thereof, analogs thereof, derivatives thereof, synthetic forms thereof, alone or in combinations thereof. In certain embodiments the active agent is highly purified. In certain embodiments the active agent is present as a highly purified extract of active agent which comprises at least 90%, 91%, 92%, 93%, 94%, 95%, 96%, 97%, 98%, 99%, 99.5%, or 99.75% (w/w) of the formulation.

In certain embodiments the active agent is provided at a concentration equal to or greater than about 90%, 91%, 92%, 93%, 94%, 95%, 96%, 97%, 98%, 99%, 99.5%, 99.75%, or 100% (w/w).

In certain embodiments the active agent is 100% synthetic. In certain embodiments the active agent has a purity equal to or greater than about 90%, 91%, 92%, 93%, 94%, 95%, 96%, 97%, 98%, 99%, 99.5%, 99.75%, or 100% (w/w). In certain embodiments the active agent is produced synthetically and has a purity equal to or greater than about 90%, 91%, 92%, 93%, 94%, 95%, 96%, 97%, 98%, 99%, 99.5%, 99.75%, or 100% (w/w). In certain embodiments the active agent is a combination of active agents, and each active agent may be produced synthetically and independently have a purity equal to or greater than about 90%, 91%, 92%, 93%, 94%, 95%, 96%, 97%, 98%, 99%, 99.5%, 99.75%, or 100% (w/w).

In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, or 45 mg/kg/day. In certain embodiments, the active agent is titrated from 5 to 20-25 mg/kg/day and optionally maintained for 10-15 days. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg/day. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 microgram. In certain embodiments, the dose of active agent is equal to or greater than, for example, about 0.01, 0.1, 0.25, 0.5, 1, 2, 3, 4, 5, 6, 7, 8, 9, 10, 15, 20, 25, 30, 35, 40, 45, 50, 55, 60, 65, 70, 75, 80, 85, 90, 95, 100, 125, 150, 175, 200, 225, 250, 275, 300, 325, 350, 375, or 400 mg. In exemplary embodiments, formulations of the disclosure may comprise active agent at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, about 80%, about 81%, about 82%, about 83%, about 84%, about 85%, about 86%, about 87%, about 88%, about 89%, about 90%, about 91%, about 92%, about 93%, about 94%, about 95%, about 96%, about 97%, about 98%, about 99%, and about 99.5% of the formulation. In exemplary embodiments, formulations of the disclosure may comprise active agent at a concentration of about 0.1 to about 50%, about 1 to 20%, of about 5% to 25%, about 10% to about 20%, or about 15% to about 18%, about 30% to about 70%, about 35% to about 65%, about 63.13%, about 40% to about 64% w/w, about 95 to about 98%, or about 95 to about 97%. In exemplary formulations of the disclosure, the active agent will represent approximately about 10% to about 10%, about 10% to about 9%, about 10% to about 8%, about 10% to about 7%, about 1% to about 6%, about 1% to about 5%, about 0.1 wt % to about 50% wt %, 1 wt % to 75 wt %, preferably 2 wt % to 30 wt %, more preferably 5 wt. % to 20 wt. % of the formulation.

The present disclosure provides compositions and methods that have the unique benefit of providing treatment of a condition as set forth herein that will allow a patient or heath care provider to titrate dose to the most effective dose per patient with the use, such as the addition, of an incremental dose patch.

The present disclosure provides compositions and methods which have the unique ability to immediately stop drug administration by simply removing the patch. For example, a patient may start with a dose that has the onset of unwanted negative side effects. The patient or heath care provider can control or stop administration (e.g., remove the patch, or titrate the administration with a lower dose patch) before it gets worse, and also dramatically shorten the time of the unwanted experience.

As used herein, the term “pharmaceutically acceptable salts” includes acid addition salts or addition salts of free bases. The term “pharmaceutically acceptable salts” of the active agent within its scope all the possible isomers and their mixtures, and any pharmaceutically acceptable metabolite, bioprecursor and/or pro-drug, such as, for example, a compound which has a structural formula different from the one of the compounds of the disclosure, and yet is directly or indirectly converted in vivo into a compound of the disclosure, upon administration to a subject, such as a mammal, particularly a human being. As used herein, the term “transdermal delivery” means delivery of drug into systemic circulation through the skin.

### Neoplasia

The invention provides a method for treating neoplasia in a patient in need of such treatment, comprising: administering to the patient therapeutically effective amounts of, for example, tetrahydrocannabinol (THC), cannabidiol (CBD), and combinations thereof, as exemplified herein, optionally in further combination with, for example, a neoplasia treating agent. The term “neoplasia” as used herein refers also to cancer, tumors, proliferative diseases, malignancies, and their metastases.

Cancer is an abnormal growth of cells which tend to proliferate in an uncontrolled way and, in some cases, to metastasize (spread). Cancer is not one disease. It is a group of more than 100 different and distinctive diseases. Cancer can involve any tissue of the body and have many different forms in each body area. Most cancers are named for the type of cell or organ in which they start. A tumor can be cancerous or benign. A benign tumor means the tumor can grow but does not spread. A cancerous tumor is malignant, meaning it can grow and spread to other parts of the body. If a cancer spreads (metastasizes), the new tumor bears the same name as the original (primary) tumor. The frequency of a particular cancer may depend on gender. While skin cancer is the most common type of malignancy for both men and women, the second most common type in men is prostate cancer and in women, breast cancer.

Examples for cancer diseases are Adenocarcinomas of the head and neck (including salivary glands and oral cavity), gastrointestinal tract (including pharynx, esophagus, stomach, small intestine, large intestine, anus), lung, liver (including hepatocellular carcinoma, cholangiocarcinoma, and mixed tumors), extrahepatic biliary tract and gallbladder, pancreas (including ductal and acinar types), genitourinary tracts (ovaries, fallopian tubes, endometrium, cervix, and vagina, ureters, urinary bladder, testicles, epididymis, prostate), and skin adnexa; squamous cell carcinomas of the head and neck (including salivary glands and oral cavity), gastrointestinal tract (including pharynx, esophagus, anus), lung, intrahepatic and extrahepatic biliary tree (including gallbladder), pancreas, genitourinary tracts (including endometrium, cervix, vagina, ureters, urinary bladder, testicles, epididymis, prostate), and skin adnexa; germ cell tumors (including malignant teratoma, embryonal carcinoma, struma ovarii, yolk sac tumor, seminoma, choriocarcinoma); sarcomas (including leiomyosarcomas, rhabdomyosarcomas, angiosarcomas, hemangioendotheliomas, liposarcomas, chondosarcomas, fibrosarcomas, Ewing sarcoma, malignant nerve sheathe tumors, alveolar soft part sarcomas, clear cell sarcomas, synovial sarcoma, osteosarcomas); malignancies of the central nervous system (including astrocytomas, oligodendroglioma, glioblastoma, GBM, medulloblastoma); salivary gland malignancies (including adenoid cystic carcinoma, adenosquamous carcinoma, clear cell carcinoma, cystadenocarcinoma, mucoepidermoid carcinoma); mixed type carcinomas (including hepatocellular-cholangiocarcinomas, carcinosarcomas, mixed adenoneurondocrine carcinomas, adenosquamous carcinomas); hepatocellular carcinoma; blastic malignancies (including hepatoblastoma, neuroblastoma, ganglioneuroblastoma, nephroblastoma); renal cell carcinomas; neuroendocrine carcinomas; thyroid carcinomas (including papillary, follicular, medullary, anaplastic carcinomas); parathyroid carcinomas, pituitary gland carcinomas, adrenal gland carcinomas (including adrenocortical carcinomas, pheochromocytoma), and combinations thereof. In some embodiments of the present disclosure, the cancer is endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and a hematological cancer, such as multiple myeloma, B-cell lymphoma, T-cell lymphoma, Hodgkin's lymphoma/primary mediastinal B-cell lymphoma, or chronic myelogenous leukemia.

The methods of the disclosure can be used to treat any type of cancer known in the art. Non-limiting examples of cancers to be treated by the methods of the present disclosure can include melanoma (e.g., metastatic malignant melanoma), renal cancer (e.g. clear cell carcinoma), prostate cancer (e.g. hormone refractory prostate adenocarcinoma), pancreatic adenocarcinoma, breast cancer, colon cancer, lung cancer (e.g. non-small cell lung cancer), esophageal cancer, squamous cell carcinoma, liver cancer, ovarian cancer, cervical cancer, thyroid cancer, glioblastoma, GBM, glioma, leukemia, lymphoma, mesothelioma, sarcoma and other neoplastic malignancies. Additionally, the invention includes refractory or recurrent malignancies whose growth may be inhibited using the methods of the invention. In some embodiments, a cancer to be treated by the methods of the present disclosure include, for example, carcinoma, squamous carcinoma (for example, cervical canal, eyelid, tunica conjunctiva, vagina, lung, oral cavity, skin, urinary bladder, head and neck, tongue, larynx, and gullet), and adenocarcinoma (for example, prostate, small intestine, endometrium, cervical canal, large intestine, lung, pancreas, gullet, rectum, uterus, stomach, mammary gland, and ovary). In some embodiments, a cancer to be treated by the methods of the present disclosure further include sarcomata (for example, myogenic sarcoma), leukosis, neuroma, melanoma, and lymphoma.

In some embodiments, a patient or population of patients to be treated with combination therapy of the present disclosure have a solid tumor. In some embodiments, a solid tumor is a melanoma, renal cell carcinoma, lung cancer, bladder cancer, breast cancer, cervical cancer, colon cancer, gall bladder cancer, laryngeal cancer, liver cancer, thyroid cancer, stomach cancer, salivary gland cancer, prostate cancer, pancreatic cancer, mesothelioma, sarcoma or Merkel cell carcinoma. In some embodiments, a patient or population of patients to be treated with combination therapy of the present disclosure have a hematological cancer. In some embodiments, the patient has a hematological cancer such as diffuse large B cell lymphoma (“DLBCL”), Hodgkin's lymphoma (“HL”), Non-Hodgkin's lymphoma (“NHL”), Follicular lymphoma (“FL”), acute myeloid leukemia (“AML”), or Multiple myeloma (“MM”).

### Neoplasia Therapies

Any therapy (e.g., therapeutic or prophylactic agent) which is useful, has been used, is currently being used, or may be used for the prevention, treatment and/or management of neoplasia can be used to prevent, treat, and/or manage the patient whose neoplasia and/or cancer in accordance with the methods, compositions, and combinations of the invention. Also, neoplasia and/or cancer monitoring can be employed in conjunction with any therapy for cancer according to the instant invention. Therapies (e.g., therapeutic or prophylactic agents) include, but are not limited to, peptides, polypeptides, fusion proteins, nucleic acid molecules, small molecules, mimetic agents, synthetic drugs, inorganic molecules, and organic molecules. Non-limiting examples of cancer therapies include chemotherapies, radiation therapies, hormonal therapies, anti-angiogenesis therapies, targeted therapies, and/or biological therapies including immunotherapies and surgery. In certain embodiments, a prophylactically and/or therapeutically effective regimen comprises the administration of a combination of therapies.

Examples of neoplasia agents or therapies include, but are not limited to: acivicin; aclarubicin; acodazole hydrochloride; acronine; adozelesin; aldesleukin; altretamine; ambomycin; ametantrone acetate; aminoglutethimide; amsacrine; anastrozole; anthracyclin; anthramycin; asparaginase; asperlin; azacitidine (Vidaza); azetepa; azotomycin; batimastat; benzodepa; bicalutamide; bisantrene hydrochloride; bisnafide dimesylate; bisphosphonates (e.g., pamidronate (Aredria), sodium clondronate (Bonefos), zoledronic acid (Zometa), alendronate (Fosamax), etidronate, ibandomate, cimadronate, risedromate, and tiludromate); bizelesin; bleomycin sulfate; brequinar sodium; bropirimine; busulfan; cactinomycin; calusterone; caracemide; carbetimer; carboplatin; carmustine; carubicin hydrochloride; carzelesin; cedefingol; chlorambucil; cirolemycin; cisplatin; cladribine; crisnatol mesylate; cyclophosphamide; cytarabine (Ara-C); dacarbazine; dactinomycin; daunorubicin hydrochloride; decitabine (Dacogen); demethylation agents, dexormaplatin; dezaguanine; dezaguanine mesylate; diaziquone; docetaxel; doxorubicin; doxorubicin hydrochloride; droloxifene; droloxifene citrate; dromostanolone propionate; duazomycin; edatrexate; eflomithine hydrochloride; EphA2 inhibitors; elsamitrucin; enloplatin; enpromate; epipropidine; epirubicin hydrochloride; erbulozole; esorubicin hydrochloride; estramustine; estramustine phosphate sodium; etanidazole; etoposide; etoposide phosphate; etoprine; fadrozole hydrochloride; fazarabine; fenretinide; floxuridine; fludarabine phosphate; fluorouracil; fluorocitabine; fosquidone; fostriecin sodium; gemcitabine; gemcitabine hydrochloride; histone deacetylase inhibitors (HDAC-Is) hydroxyurea; idarubicin hydrochloride; ifosfamide; ilmofosine; imatinib mesylate (Gleevec, Glivec); interleukin II (including recombinant interleukin II, or rIL2), interferon alpha-2a; interferon alpha-2b; interferon alpha-n1; interferon alpha-n3; interferon beta-I a; interferon gamma-I b; iproplatin; irinotecan hydrochloride; lanreotide acetate; lenalidomide (Revlimid); letrozole; leuprolide acetate; liarozole hydrochloride; lometrexol sodium; lomustine; losoxantrone hydrochloride; masoprocol; maytansine; mechlorethamine hydrochloride; anti-CD2 antibodies (e.g., siplizumab (MedImmune Inc.; International Publication No. WO 02/098370, which is incorporated herein by reference in its entirety)); megestrol acetate; melengestrol acetate; melphalan; menogaril; mercaptopurine; methotrexate; methotrexate sodium; metoprine; meturedepa; mifepristone; mitindomide; mitocarcin; mitocromin; mitogillin; mitomalcin; mitomycin; mitosper; mitotane; mitoxantrone hydrochloride; mycophenolic acid; nocodazole; nogalamycin; ormaplatin; oxaliplatin; oxisuran; paclitaxel; pegaspargase; peliomycin; pentamustine; peplomycin sulfate; perfosfamide; pipobroman; piposulfan; piroxantrone hydrochloride; plicamycin; plomestane; porfimer sodium; porfiromycin; prednimustine; procarbazine hydrochloride; puromycin; puromycin hydrochloride; pyrazofurin; riboprine; rogletimide; RU486; safingol; safingol hydrochloride; semustine; simtrazene; sparfosate sodium; sparsomycin; spirogermanium hydrochloride; spiromustine; spiroplatin; streptonigrin; streptozocin; sulofenur; talisomycin; tecogalan sodium; tegafur; teloxantrone hydrochloride; temoporfin; teniposide; teroxirone; testolactone; thiamiprine; thioguanine; thiotepa; tiazofurin; tirapazamine; toremifene citrate; trestolone acetate; triciribine phosphate; trimetrexate; trimetrexate glucuronate; triptorelin; tubulozole hydrochloride; uracil mustard; uredepa; vapreotide; verteporfin; vinblastine sulfate; vincristine sulfate; vindesine; vindesine sulfate; vinepidine sulfate; vinglycinate sulfate; vinleurosine sulfate; vinorelbine tartrate; vinrosidine sulfate; vinzolidine sulfate; vorozole; zeniplatin; zinostatin; zorubicin hydrochloride.

Other examples of cancer therapies include, but are not limited to: 20-epi-1,25 dihydroxyvitamin D3; 5-ethynyluracil; abiraterone; aclarubicin; acylfulvene; adecypenol; adozelesin; aldesleukin; ALL-TK antagonists; altretamine; ambamustine; amidox; amifostine; aminolevulinic acid; amrubicin; amsacrine; anagrelide; anastrozole; andrographolide; angiogenesis inhibitors; antagonist D; antagonist G; antarelix; anti-dorsalizing morphogenetic protein-1; antiandrogen, prostatic carcinoma; antiestrogen; antineoplaston; antisense oligonucleotides; aphidicolin glycinate; apoptosis gene modulators; apoptosis regulators; apurinic acid; ara-CDP-DL-PTBA; arginine deaminase; asulacrine; atamestane; atrimustine; axinastatin 1; axinastatin 2; axinastatin 3; azasetron; azatoxin; azatyrosine; baccatin III derivatives; balanol; batimastat; BCR/ABL antagonists; benzochlorins; benzoylstaurosporine; beta lactam derivatives; beta-alethine; betaclamycin B; betulinic acid; bFGF inhibitor; bicalutamide; bisantrene; bisaziridinylspermine; bisnafide; bistratene A; bizelesin; breflate; bropirimine; budotitane; buthionine sulfoximine; calcipotriol; calphostin C; camptothecin derivatives; canarypox IL-2; capecitabine; carboxamide-amino-triazole; carboxyamidotriazole; CaRest M3; CARN 700; cartilage derived inhibitor; carzelesin; casein kinase inhibitors (ICOS); castanospermine; cecropin B; cetrorelix; chlorins; chloroquinoxaline sulfonamide; cicaprost; cis-porphyrin; cladribine; clomifene analogues; clotrimazole; collismycin A; collismycin B; combretastatin A4; combretastatin analogue; conagenin; crambescidin 816; crisnatol; cryptophycin 8; cryptophycin A derivatives; curacin A; cyclopentanthraquinones; cycloplatam; cypemycin; cytarabine ocfosfate; cytolytic factor; cytostatin; dacliximab; decitabine; dehydrodidemnin B; deslorelin; dexamethasone; dexifosfamide; dexrazoxane; dexverapamil; diaziquone; didemnin B; didox; diethylnorspermine; dihydro-5-azacytidine; dihydrotaxol, dioxamycin; diphenyl spiromustine; docetaxel; docosanol; dolasetron; doxifluridine; droloxifene; dronabinol; duocarmycin SA; ebselen; ecomustine; edelfosine; edrecolomab; eflomithine; elemene; emitefur; epirubicin; epristeride; estramustine analogue; estrogen agonists; estrogen antagonists; etanidazole; etoposide phosphate; exemestane; fadrozole; fazarabine; fenretinide; filgrastim; finasteride; flavopiridol; flezelastine; fluasterone; fludarabine; fluorodaunorunicin hydrochloride; forfenimex; formestane; fostriecin; fotemustine; gadolinium texaphyrin; gallium nitrate; galocitabine; ganirelix; gelatinase inhibitors; gemcitabine; glutathione inhibitors; HMG CoA reductase inhibitors (e.g., atorvastatin, cerivastatin, fluvastatin, lescol, lupitor, lovastatin, rosuvastatin, and simvastatin); hepsulfam; heregulin; hexamethylene bisacetamide; hypericin; ibandronic acid; idarubicin; idoxifene; idramantone; ilmofosine; ilomastat; imidazoacridones; imiquimod; immunostimulant peptides; insulin-like growth factor-1 receptor inhibitor; interferon agonists; interferons; interleukins; iobenguane; iododoxorubicin; ipomeanol, 4-iroplact; irsogladine; isobengazole; isohomohalicondrin B; itasetron; jasplakinolide; kahalalide F; lamellarin-N triacetate; lanreotide; leinamycin; lenograstim; lentinan sulfate; leptolstatin; letrozole; leukemia inhibiting factor; leukocyte alpha interferon; leuprolide+estrogen+progesterone; leuprorelin; levamisole; LFA-3TIP (Biogen, Cambridge, Mass.; International Publication No. WO 93/0686 and U.S. Pat. No. 6,162,432); liarozole; linear polyamine analogue; lipophilic disaccharide peptide; lipophilic platinum compounds; lissoclinamide 7; lobaplatin; lombricine; lometrexol; lonidamine; losoxantrone; lovastatin; loxoribine; lurtotecan; lutetium texaphyrin; lysofylline; lytic peptides; maitansine; mannostatin A; marimastat; masoprocol; maspin; matrilysin inhibitors; matrix metalloproteinase inhibitors; menogaril; merbarone; meterelin; methioninase; metoclopramide; MIF inhibitor; mifepristone; miltefosine; mirimostim; mismatched double stranded RNA; mitoguazone; mitolactol; mitomycin analogues; mitonafide; mitotoxin fibroblast growth factor-saporin; mitoxantrone; mofarotene; molgramostim; monoclonal antibody, human chorionic gonadotrophin; monophosphoryl lipid A+myobacterium cell wall sk; mopidamol; multiple drug resistance gene inhibitor; multiple tumor suppressor 1-based therapy; mustard anticancer agent; mycaperoxide B; mycobacterial cell wall extract; myriaporone; N-acetyldinaline; N-substituted benzamides; nafarelin; nagrestip; naloxone+pentazocine; napavin; naphterpin; nartograstim; nedaplatin; nemorubicin; neridronic acid; neutral endopeptidase; nilutamide; nisamycin; nitric oxide modulators; nitroxide antioxidant; nitrullyn; O6-benzylguanine; octreotide; okicenone; oligonucleotides; onapristone; oracin; oral cytokine inducer; ormaplatin; osaterone; oxaliplatin; oxaunomycin; paclitaxel; paclitaxel analogues; paclitaxel derivatives; palauamine; palmitoylrhizoxin; pamidronic acid; panaxytriol; panomifene; parabactin; pazelliptine; pegaspargase; peldesine; pentosan polysulfate sodium; pentostatin; pentrozole; perflubron; perfosfamide; perillyl alcohol; phenazinomycin; phenylacetate; phosphatase inhibitors; picibanil; pilocarpine hydrochloride; pirarubicin; piritrexim; placetin A; placetin B; plasminogen activator inhibitor; platinum complex; platinum compounds; platinum-triamine complex; porfimer sodium; porfiromycin; prednisone; propyl bis-acridone; prostaglandin J2; proteasome inhibitors; protein A-based immune modulator; protein kinase C inhibitor; protein kinase C inhibitors, microalgal; protein tyrosine phosphatase inhibitors; purine nucleoside phosphorylase inhibitors; purpurins; pyrazoloacridine; pyridoxylated hemoglobin polyoxyethylene; raf antagonists; raltitrexed; ramosetron; ras farnesyl protein transferase inhibitors; ras inhibitors; ras-GAP inhibitor; retelliptine demethylated; rhenium Re 186 etidronate; rhizoxin; ribozymes; RII retinamide; rogletimide; rohitukine; romurtide; roquinimex; rubiginone B1; ruboxyl; safingol; saintopin; SarCNU; sarcophytol A; sargramostim; Sdi 1 mimetics; semustine; senescence derived inhibitor 1; sense oligonucleotides; signal transduction inhibitors; signal transduction modulators; gamma secretase inhibitors, single chain antigen binding protein; sizofuran; sobuzoxane; sodium borocaptate; sodium phenylacetate; solverol; somatomedin binding protein; sonermin; sparfosic acid; spicamycin D; spiromustine; splenopentin; spongistatin 1; squalamine; stem cell inhibitor; stem-cell division inhibitors; stipiamide; stromelysin inhibitors; sulfinosine; superactive vasoactive intestinal peptide antagonist; suradista; suramin; swainsonine; synthetic glycosaminoglycans; tallimustine; 5-fluorouracil; leucovorin; tamoxifen methiodide; tauromustine; tazarotene; tecogalan sodium; tegafur; tellurapyrylium; telomerase inhibitors; temoporfin; temozolomide; teniposide; tetrachlorodecaoxide; tetrazomine; thaliblastine; thiocoraline; thrombopoietin; thrombopoietin mimetic; thymalfasin; thymopoietin receptor agonist; thymotrinan; thyroid stimulating hormone; tin ethyl etiopurpurin; tirapazamine; titanocene bichloride; topsentin; toremifene; totipotent stem cell factor; translation inhibitors; tretinoin; triacetyluridine; triciribine; trimetrexate; triptorelin; tropisetron; turosteride; tyrosine kinase inhibitors; tyrphostins; UBC inhibitors; ubenimex; urogenital sinus-derived growth inhibitory factor; urokinase receptor antagonists; vapreotide; variolin B; vector system, erythrocyte gene therapy; thalidomide; velaresol; veramine; verdins; verteporfin; vinorelbine; vinxaltine; anti-integrin antibodies (e.g., anti-integrin a.sub.vb.sub.3 antibodies); vorozole; zanoterone; zeniplatin; zilascorb; and zinostatin stimalamer.

A non-limiting list of compounds that could be used to target neoplasia and/or cancer ncludes: inhibitors of interleukin-3 receptor (IL-3R) and CD 123 (including peptides, peptide-conjugates, antibodies, antibody-conjugates, antibody fragments, and antibody fragment-conjugates that target IL-3R or CD123); cantharidin; norcantharidin and analogs and derivatives thereof, Notch pathway inhibitors including gamma secretase inhibitors; sonic hedgehog/smoothened pathway inhibitors including cyclopamine and analogs thereof, antibodies to CD96; certain NF-kB/proteasome inhibitors including parthenolide and analogs thereof, certain triterpenes including celastrol; certain mTOR inhibitors; compounds and antibodies that target the urokinase receptor; sinefungin; certain inosine monophosphate dehydrogenase (IMPDH) inhibitors; PPAR-alpha and PPAR-gamma agonists and antagonists (including pioglitazone, tesaslitazar, muraglitazar, peliglitazar, lobeglitazone, balaglitazone, ragaglitazar, rosiglitazone, farglitazar, sodelglitazar, reglitazar, naveglitazar, oxeglitazar, metaglidasen, netoglitazone, darglitazone, englitazone, thiazolidinediones, aleglitazar, edaglitazone, rivoglitazone, troglitazone, imiglitazar, and sipoglitazar); telomerase inhibitors; antibodies to EpCAM (ESA); GSK-3 beta agonists and antagonists (including Lithium, 6-bromoinirubin-3′-oxime (BIO), TDZD8); Wnt pathway inhibitors including antibodies to frizzled or small molecules that inhibit disheveled/frizzled or beta catenin; anti-CD20 antibodies and conjugates (e.g. Rituxan, Bexxar, Zevalin) for novel use in multiple myeloma or melanoma; anti-CD133 antibody; anti-CD44 antibody; antibodies to IL-4; certain differentiation agents such as versnarinone; compounds that target CD33 such as an antibody or betulinic acid; compounds that target lactadherin such as an antibody; small molecules or antibodies that target CXCR4 or SDF-1; small molecules or antibodies that target multi-drug resistance pumps; inhibitors of survivin; inhibitors of XIAP; small molecules that target Bcl-2; antibodies to CLL-1; and furin inhibitors (such as cucurbitacins).

An additional non-limiting list of compounds that could also be used to target cancer and/or cancer cells includes: i) antibodies, antibody fragments, and proteins that are either naked or conjugated to a therapeutic moiety that target certain cell surface targets on cancer cells, or ii) small molecules known in the art including ones that can be further optimized (e.g., via chemistry) or identified via a cancer cell-based screen (e.g., such as one that would determine whether a compound impairs proliferation or viability of a cancer cell through standard methods, the cell surface and intracellular targets including (not meant to be exhaustive) are: Rex1 (Zfp42), CTGF, Activin A, Wnt, FGF-2, HIF-1, AP-2gamma, Bmi-1, nucleostemin, hiwi, Moz-TIF2, Nanog, beta-arrestin-2, Oct-4, Sox2, stella, GDF3, RUNX3, EBAF, TDGF-1, nodal, ZFPY, PTNE, Evi-1, Pax3, Mcl-1, c-kit, Lex-1, Zfx, lactadherin, aldehyde dehydrogenase, BCRP, telomerase, CD133, Bcl-2, CD26, Gremlin, and FoxC2.

In some embodiments, the therapy(ies) is an immunomodulatory agent. Non-limiting examples of immunomodulatory agents include proteinaceous agents such as cytokines, peptide mimetics, and antibodies (e.g., human, humanized, chimeric, monoclonal, polyclonal, Fvs, ScFvs, Fab or F(ab)2 fragments or epitope binding fragments), nucleic acid molecules (e.g., antisense nucleic acid molecules and triple helices), small molecules, organic compounds, and inorganic compounds. In particular, immunomodulatory agents include, but are not limited to, methotrexate, leflunomide, cyclophosphamide, cytoxan, Immuran, cyclosporine A, minocycline, azathioprine, antibiotics (e.g., FK506 (tacrolimus)), methylprednisolone (MP), corticosteroids, steroids, mycophenolate mofetil, rapamycin (sirolimus), mizoribine, deoxyspergualin, brequinar, malononitriloamides (e.g., leflunamide), T cell receptor modulators, cytokine receptor modulators, and modulators mast cell modulators. Other examples of immunomodulatory agents can be found, e.g., in U.S. Publication No. 2005/0002934 A1 at paragraphs 259-275 which is incorporated herein by reference in its entirety. In one embodiment, the immunomodulatory agent is a chemotherapeutic agent. In an alternative embodiment, the immunomodulatory agent is an immunomodulatory agent other than a chemotherapeutic agent. In some embodiments, the therapy(ies) used in accordance with the invention is not an immunomodulatory agent.

In some embodiments, the therapy(ies) is an anti-angiogenic agent. Non-limiting examples of anti-angiogenic agents include proteins, polypeptides, peptides, fusion proteins, antibodies (e.g., human, humanized, chimeric, monoclonal, polyclonal, Fvs, ScFvs, Fab fragments, F(ab2 fragments, and antigen-binding fragments thereof) such as antibodies that specifically bind to TNF-alpha, nucleic acid molecules (e.g., antisense molecules or triple helices), organic molecules, inorganic molecules, and small molecules that reduce or inhibit angiogenesis.

In certain embodiments, the therapy(ies) is an alkylating agent, a nitrosourea, an antimetabolite, and anthracyclin, a topoisomerase II inhibitor, or a mitotic inhibitor. Alkylating agents include, but are not limited to, busulfan, cisplatin, carboplatin, chlorambucil, cyclophosphamide, ifosfamide, decarbazine, mechlorethamine, mephalen, and themozolomide. Nitrosoureas include, but are not limited to carmustine (BCNU) and lomustine (CCNU). Antimetabolites include but are not limited to 5-fluorouracil, capecitabine, methotrexate, gemcitabine, cytarabine, and fludarabine. Anthracyclins include but are not limited to daunorubicin, doxorubicin, epirubicin, idarubicin, and mitoxantrone. Topoisomerase II inhibitors include, but are not limited to, topotecan, irinotecan, etopiside (VP-16), and teniposide. Mitotic inhibitors include, but are not limited to taxanes (paclitaxel, docetaxel), and the vinca alkaloids (vinblastine, vincristine, and vinorelbine). In some embodiments of the invention, the therapy(ies) includes the administration cantharidin or an analog thereof. The invention includes the use of agents that target cancer cells. In certain embodiments, the agent acts alone. In other embodiments, the agent is attached directly or indirectly to another therapeutic moiety. Non-limiting examples of therapeutic moieties include, but are not limited to alkylating agents, anti-metabolites, plant alkaloids, cytotoxic agents, chemotherapeutic agents (e.g., a steroid, cytosine arabinoside, fluoruracil, methotrexate, aminopterin, mitomycin C, demecolcine, etoposide, mithramycin, calicheamicin, CC-1065, chlorambucil or melphalan), radionuclides, therapeutic enzymes, cytokines, toxins including plant-derived toxins, fungus-derived toxins, bacteria-derived toxin (e.g., deglycosylated ricin A chain, a ribosome inactivating protein, alpha-sarcin, aspergillin, restirictocin, a ribonuclease, a diphtheria toxin, Pseudomonas exotoxin, a bacterial endotoxin or the lipid A moiety of a bacterial endotoxin), growth modulators and RNase. In some embodiments, the agent used is an agent that binds to a marker, e.g., an antigen on a cancer cell. In a specific embodiment, the agent binds to an antigen that is expressed at a greater level on cancer cells than on normal cells. In a specific embodiment, the agent binds specifically to a cancer cell antigen that is not a normal cell. In other embodiments, the therapy(ies) is an agent that binds to a marker on cancer cells. In one embodiment, the agent that binds to a marker on cancer cells is an antibody or an antibody conjugated to a therapeutic moiety or an antibody fragment conjugated to a therapeutic moiety.

For example, in a specific embodiment, the agent binds specifically to the IL-3 Receptor (IL-3R). In some embodiments, the agent that binds to the IL-3R is an antibody or an antibody fragment that is specific for IL-3R. In some embodiments, the antibody or antibody fragment is conjugated either chemically or via recombinant technology to a therapeutic moiety (e.g., a chemotherapeutic agent, a plant-, fungus- or bacteria-derived toxin, a radionuclide) using a linking agent to effect a cell killing response. In certain embodiments, the antibody, antibody-conjugate, antibody fragment, or antibody fragment-conjugate binds to the .alpha.-subunit of IL-3R (i.e., the CD 123 antigen). In other embodiments, the antibody, antibody-conjugate, antibody fragment, or antibody fragment-conjugate binds to the IL-3R, containing both the alpha and beta subunits. Methods for preparing antibodies to IL-3R and mimetics of antibodies to IL-3R are described in U.S. Pat. No. 6,733,743 B2, which is incorporated herein by reference in its entirety.

In other embodiments, the agent that binds to a marker on cancer cells is a ligand. In some embodiments, the ligand is a cytokine that binds to a cytokine receptor on cancer cells. In a particular embodiment, the ligand is interleukin-3 (IL-3) which can be conjugated to a therapeutic moiety that includes a chemotherapeutic agent, a plant-, fungus-, or bacteria-derived toxin, or a radionuclide. The IL-3-conjugate prophylactic and/or therapeutic therapy or regimen can be in the form of a recombinant fusion protein in embodiments where the conjugate is a toxin and the toxin is a protein, such as diphtheria toxin.

In certain embodiments, antibodies or fragments thereof that bind to a marker on cancer cells are substantially non-immunogenic in the treated subject. Methods for obtaining non-immunogenic antibodies include, but are not limited to, chimerizing the antibody, humanizing the antibody, and isolating antibodies from the same species as the subject receiving the therapy. Antibodies or fragments thereof that bind to markers in cancer cells can be produced using techniques known in the art.

In some embodiments, the therapy comprises the use of X-rays, gamma rays and other sources of radiation to destroy cancer cells and/or cancer cells. In specific embodiments, the radiation therapy is administered as external beam radiation or teletherapy, wherein the radiation is directed from a remote source. In other embodiments, the radiation therapy is administered as internal therapy or brachytherapy wherein a radioactive source is placed inside the body close to cancer cells, cancer cells and/or a tumor mass.

In some embodiments, the therapy used is a proliferation based therapy. Non-limiting examples of such therapies include a chemotherapy and radiation therapy as described supra.

Currently available therapies and their dosages, routes of administration and recommended usage are known in the art and have been described in such literature as the Physician's Desk Reference (60th ed., 2006).

When two prophylactically and/or therapeutically effective regimens are administered to a subject concurrently, the term “concurrently” is not limited to the administration of the cancer therapeutics at exactly the same time, but rather, it is meant that they are administered to a subject in a sequence and within a time interval such that they can act together (e.g., synergistically to provide an increased benefit than if they were administered otherwise). For example, the cancer therapeutics may be administered at the same time or sequentially in any order at different points in time; however, if not administered at the same time, they should be administered sufficiently close in time so as to provide the desired therapeutic effect, preferably in a synergistic fashion. The combination cancer therapeutics can be administered separately, in any appropriate form and by any suitable route. When the components of the combination cancer therapeutics are not administered in the same pharmaceutical composition, it is understood that they can be administered in any order to a subject in need thereof. For example, a first prophylactically and/or therapeutically effective regimen can be administered prior to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks before), concomitantly with, or subsequent to (e.g., 5 minutes, 15 minutes, 30 minutes, 45 minutes, 1 hour, 2 hours, 4 hours, 6 hours, 12 hours, 24 hours, 48 hours, 72 hours, 96 hours, 1 week, 2 weeks, 3 weeks, 4 weeks, 5 weeks, 6 weeks, 8 weeks, or 12 weeks after) the administration of the second cancer therapeutic, to a subject in need thereof. In various embodiments, the cancer therapeutics are administered 1 minute apart, 10 minutes apart, 30 minutes apart, less than 1 hour apart, 1 hour apart, 1 hour to 2 hours apart, 2 hours to 3 hours apart, 3 hours to 4 hours apart, 4 hours to 5 hours apart, 5 hours to 6 hours apart, 6 hours to 7 hours apart, 7 hours to 8 hours apart, 8 hours to 9 hours apart, 9 hours to 10 hours apart, 10 hours to 11 hours apart, 11 hours to 12 hours apart, no more than 24 hours apart or no more than 48 hours apart. In one embodiment, the cancer therapeutics are administered within the same office visit. In another embodiment, the combination cancer therapeutics are administered at 1 minute to 24 hours apart.

In a specific embodiment, the combination therapies have the same mechanism of action. In another specific embodiment, the combination therapies each have a different mechanism of action.

### Additional Active Agents

As used herein the term “combination administration” of a compound, therapeutic agent or known drug with the combination of the present invention means administration of the drug and the one or more compounds at such time that both the known drug and/or combination will have a therapeutic effect. In some cases this therapeutic effect will be synergistic. Such concomitant administration can involve concurrent (i.e. at the same time), prior, or subsequent administration of the drug with respect to the administration of the composition and/or combination of the present invention. A person of ordinary skill in the art would have no difficulty determining the appropriate timing, sequence and dosages of administration for particular drugs of the present invention.

Further, active ingredient(s), where applicable, may be present either in the form of one substantially optically pure enantiomer or as a mixture of enantiomers or polymorphs thereof.

The active ingredient(s) may comprise one or more of the following therapeutic classes but not limited to adrenergic agent; adrenocortical steroid; adrenocortical suppressant; aldosterone antagonist; amino acid; anabolic; analeptic; analgesic; anesthetic; anorectic; anti-acne agent; anti-adrenergic; anti-allergic; anti-amebic; anti-anemic; anti-anginal; anti-arthritic; anti-asthmatic; anti-atherosclerotic; antibacterial; anticholinergic; anticoagulant; anticonvulsant; antidepressant; antidiabetic; antidiarrheal; antidiuretic; anti-emetic; anti-epileptic; antifibrinolytic; antifungal; antihemorrhagic; antihistamine; antihyperlipidemia; antihypertensive; antihypotensive; anti-infective; anti-inflammatory; antimicrobial; antimigraine; antimitotic; antimycotic, antinauseant, antineoplastic, antineutropenic, antiparasitic; antiproliferative; antipsychotic; antirheumatic; antiseborrheic; antisecretory; antispasmodic; antithrombotic; anti-ulcerative; antiviral; appetite suppressant; blood glucose regulator; bone resorption inhibitor; bronchodilator; cardiovascular agent; cholinergic; depressant; diagnostic aid; diuretic; dopaminergic agent; estrogen receptor agonist; fibrinolytic; fluorescent agent; free oxygen radical scavenger; gastric acid suppressant; gastrointestinal motility effector; glucocorticoid; hair growth stimulant; hemostatic; histamine H2 receptor antagonists; hormone; hypocholesterolemic; hypoglycemic; hypolipidemic; hypotensive; imaging agent; immunizing agent; immunomodulator; immunoregulator; immunostimulant; immunosuppressant; keratolytic; LHRH agonist; mood regulator; mucolytic; mydriatic; nasal decongestant; neuromuscular blocking agent; neuroprotective; NMDA antagonist; non-hormonal sterol derivative; plasminogen activator; platelet activating factor antagonist; platelet aggregation inhibitor; psychotropic; radioactive agent; scabicide; sclerosing agent; sedative; sedative-hypnotic; selective adenosine Al antagonist; serotonin antagonist; serotonin inhibitor; serotonin receptor antagonist; steroid; thyroid hormone; thyroid inhibitor; thyromimetic; tranquilizer; amyotrophic lateral sclerosis agent; cerebral ischemia agent; Paget's disease agent; unstable angina agent; vasoconstrictor; vasodilator; wound healing agent; xanthine oxidase inhibitor.

As indicated the pharmaceutical formulations as disclosed herein may comprise auxiliary excipients such as for example diluents, binders, lubricants, surfactants, disintegrants, plasticizers, anti-tack agents, opacifying agents, pigments, and such like. As will be appreciated by those skilled in the art, the exact choice of excipient and their relative amounts will depend to some extent on the final oral dosage form.

### Pharmaceutical Compositions

According to certain embodiments described herein, pharmaceutical composition or transdermal formulation of contains active agents such as tetrahydrocannabinol (THC), cannabidiol (CBD), or combinations thereof, and derivatives of these compounds. More preferably transdermal formulation may include active agents such as tetrahydrocannabinol (THC), cannabidiol (CBD), or combinations thereof, and derivatives of these compounds.

One embodiment of the present disclosure can be a transdermal drug delivery system which may include without any limitation to transdermal formulation, transdermal patches, topical formulation, microneedles, iontophoresis, metered dose transdermal spray.

Transdermal formulation which includes liquids for example without any limitation like solutions, suspensions, dispersions, emulsion. Transdermal formulation includes semisolids for example without any limitations like gels, ointments, emulsions, creams, suspension, paste, lotion, balm. Liquid formulation and/or gel formulation incorporated in transdermal patch is preferred. Transdermal formulations which includes polymer matrix without any limitations like adhesive matrix, non-adhesive matrix.

Without any limitation, transdermal patch may include all transdermal drug delivery systems stated in art preferably but not limited to reservoir patch, matrix patch, bilayer matrix patch, multilayer matrix patch, microreservoir patch, adhesive systems, transdermally applicable tape and other.

In certain embodiments of the present disclosure, a transdermal patch comprises transdermal formulation containing active agents such as tetrahydrocannabinol (THC), cannabidiol (CBD), or combinations thereof, and derivatives of these compounds contained in a reservoir or a matrix, and an adhesive which allows the transdermal patch to adhere to the skin, allowing the passage of the active agents such as tetrahydrocannabinol (THC), cannabidiol (CBD), or combinations thereof, and derivatives of these compounds from the transdermal patch through the skin of the patient. The transdermal delivery system can be occlusive, semi-occlusive or non-occlusive, and can be adhesive or non-adhesive.

The transdermal formulation comprising active agents such as tetrahydrocannabinol (THC), cannabidiol (CBD), or combinations thereof, and derivatives of these compounds can be incorporated within the patch and patch can be applied topically to the skin surface. The patch can be left on the subject for any suitable period of time.

In some embodiments, the transdermal patches provide for a constant rate of delivery of the active components of the transdermal patch over a predetermined time period. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a steady absorption rate of the active components of the transdermal patches by the patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a constant blood serum level of the active components of the transdermal patches in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein provide a plasma concentration of the active components of the transdermal patches in a therapeutic range in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the transdermal patches described herein allow for reduced variability in dosage of active components in a patient over a predetermined time. In some embodiments, the predetermined time period is 24 hours, 48 hours, 72 hours, 96 hours, 120 hours, 144 hours, 7 days, 8 to 13 days, two weeks, or 15 days.

In yet further embodiments, the pharmaceutical compositions described herein provide a plasma concentration of the active agent components of the transdermal patches in a therapeutic range in a patient over a predetermined time. In exemplary embodiments as disclosed herein, the pharmaceutical composition provides a blood serum level of active agent selected from without any limitation, of, for example, about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, about 5 μg/mL, and ranges thereof. In one aspect, the pharmaceutical composition provides a blood serum level of active agent in the range of 0.01 ng/mL-400 ng/mL. In another aspect, transdermal patch provides a blood serum level of active agent in the range of 0.01 ng/mL-100 ng/mL. In yet another aspect the pharmaceutical composition provides a blood serum level of active agent in the range of from 0.01-1 ng/ml to 1-100 ng/ml to 100-500 ng/ml to 500-1000 ng/ml to 1000-5000 ng/ml.

In yet further embodiments, the pharmaceutical compositions described herein provide a plasma concentration of the active agent components of the transdermal patches in a therapeutic range in a patient over a predetermined time. In exemplary embodiments as disclosed herein, the pharmaceutical composition provides a blood serum level of active agent selected from without any limitation, of, for example, about 0.01 microgm/mL, about 0.02 microgm/mL, about 0.05 microgm/mL, about 0.1 microgm/mL, about 0.2 microgm/mL, about 0.5 microgm/mL, about 1 microgm/mL, about 2 microgm/mL, about 5 microgm/mL, about 10 microgm/mL, about 20 microgm/mL, about 50 microgm/mL, about 100 microgm/mL, about 200 microgm/mL, about 500 microgm/mL, about 1 milligm/mL, about 2 milligm/mL, about 5 milligm/mL, and ranges thereof. In one aspect, the pharmaceutical composition provides a blood serum level of active agent in the range of 0.01 microgm/mL-400 microgm/mL. In another aspect, the pharmaceutical composition provides a blood serum level of active agent in the range of 0.01 microgm/mL-100 microgm/mL. In yet another aspect the pharmaceutical composition provides a blood serum level of active agent in the range of from 0.01-1 microgm/mL to 1-100 microgm/mL to 100-500 microgm/mL to 500-1000 microgm/mL to 1000-5000 microgm/mL.

The topical formulation stated in the art which include, for example without any limitation, semisolids such as ointment, cream, emulsion, micro emulsion, nano emulsion, paste, balms, gels, lotions, mousses. Liquids such as solutions, suspensions, micro suspension, nano suspension, dispersions, nano dispersion etc. Sprays, aerosols, magma, etc. The topical formulation comprising such as tetrahydrocannabinol (THC), cannabidiol (CBD), or combinations thereof, and derivatives of these compounds can be topically applied to the skin surface for transdermal delivery of such as tetrahydrocannabinol (THC), cannabidiol (CBD), or combinations thereof, and derivatives of these compounds.

One embodiment of the present disclosure can be a topical drug delivery system which may include without any limitation to topical patches, topical formulation, metered dose topical spray, topical film forming formulation, topical drug-in-adhesive patches, topical matrix patches, topical aerosols, metered dose topical gel.

Multilayer Polymer Drug Matrix System: For instance, a transdermal drug delivery system is contemplated where the active substance matrix is constructed using water soluble polymers, which is then coated on the adhesive layer. Further, the active substance reservoir can be prepared as a polymer matrix. In addition, the active substance reservoir can be confined on the skin facing side of the transdermal drug delivery system by an active substance permeable membrane and on the opposite side from the skin by an active substance impermeable layer followed by adhesive layer.

The disclosure provides a transdermal drug delivery system comprising an active substance matrix containing area is a double or multilayered active substance matrix. In another embodiment, the active substance is in the simplest case dispersed, coarsely, colloidally or molecularly, in a solution or melt of base polymers. In the further a transdermal drug delivery system manufacturing techniques, the active agent is in the form of supersaturated solution, nano-emulsion or nano-suspension, amorphous, crystalline, co-crystals, coated with base polymers or solubilize in polymers using hot melt extrusion process.

The disclosure also includes such embodiments where the active agent matrix has a two or multi-layered structure, also called multi-laminate drug in adhesive patch. For example, the various matrix layers may contain polymer constitutes from the above-mentioned polymers. In this case, the matrix layers are differing from each other's in the term of polymer or pressure sensitive or hot melt polymers composition, active agent concentration, different permeating enhancers or solubilizers. The layers can be separated using semi-permeable membrane between two distinct drug-in-adhesive layers or multiple drug-in-adhesive layers under a single backing film. The term polymer film includes polymer without any limitation pressure sensitive adhesive and/or non-adhesive polymer.

In one aspect the disclosure further provides a polymer matrix formulation comprising active agent and a polymeric vehicle system. The vehicle system can include solvents (e.g., a solubilizer), permeability enhancing excipients and polymer or gelling agent or thickening agent, if required acid or base for pH adjustment.

Pretreatment: Various approaches have been used to open the barrier property of stratum corneum for drug permeation enhancement. Pretreatment with the use of chemical penetration enhancers is one of the techniques employed. The pretreatment has a potential to modulate the outermost layer of the skin reversibly and facilitate the drug uptake. Penetration enhancers act on lipid and protein regions in combination or alone on each region.

Penetration enhancers may be incorporated into the formulations described herein (e.g., transdermal drug delivery systems including drug in adhesive layers and separate adhesive and drug containing layers), however, it can lead to some incompatibility or interactions within the ingredients. Therefore, the present disclosure provides the alternative method of skin penetration enhancement as to preparation/pretreatment the skin with some penetration enhancers or a combination of penetration enhancers before the patch application.

Pretreatment applications described herein include application of a gel/spray/solution/wetting agent/soaked swab/soaked cotton ball/soaked gauzes to the skin prior to application of drug containing product, intended to be a patch. However, it is to be understood that the pretreatment composition can include another topical dosage form, solution gel, cream, etc. For instance, the pretreatment composition can be its own individual patch, such as CuradMediplast, a 40% salicylic acid patch, or a placebo patch comprising non-volatile components such as acrylic, silicone, or PIB adhesives or combinations thereof with an optional addition of a skin permeation enhancers to promote delivery of an active pharmaceutical ingredient through the skin.

The present disclosure provides a pretreatment composition wherein the penetration enhancers are incorporated in the form the topical dosage form as solution, gel, cream, spray, wetting agent, soaked cotton balls and gauzes. In yet another embodiment pretreatment composition preferably but not limited to gel can be incorporated in a reservoir patch.

Topical formulation which includes liquids for example without any limitation like solutions, suspensions, dispersions, emulsion. Topical formulation includes semisolids for example without any limitations like gels, ointments, emulsions, creams, suspension, paste, lotion, balm. Liquid formulation and/or gel formulation incorporated in without any limitation to topical patch, metered dose topical system, sachet, etc. Topical formulations which includes polymer matrix patch without any limitations like adhesive matrix patch, non-adhesive matrix, drug-in-adhesive matrix patch, a topical matrix formulation as drug in adhesive matrix patch is preferred. Other topical formulations include such as but not limited to topical gel, metered dose topical spray, metered dose topical aerosols, topical film forming formulation.

Without any limitation, topical patch may include all topical drug delivery systems stated in art preferably but not limited to a multilayer transdermal matrix system, reservoir patch, matrix patch, bilayer matrix patch, multilayer matrix patch, microreservoir patch, adhesive systems, topically applicable tape and other.

In certain embodiments of the present disclosure, a topical patch comprises topical formulation containing active agents such as CBD and/or THC, and derivatives of these compounds contained in a reservoir or a matrix, and an adhesive which allows the topical patch to adhere to the skin, allowing the passage of the active agents such as CBD and/or THC, and derivatives of these compounds from the topical patch to the skin of the patient. The topical delivery system can be occlusive, semi-occlusive or non-occlusive, and can be adhesive or non-adhesive.

The topical formulation comprising active agents such as CBD and/or THC, and derivatives of these compounds can be incorporated within the patch and patch can be applied topically to the skin surface. The patch can be left on the subject for any suitable period of time.

The transdermal formulation and/or topical formulation of some embodiments of the present disclosure may include carriers or ingredients in effective amount either alone or in combinations thereof without any limitation to the following carriers or ingredients such as solvents, gelling agents, polymers, biodegradable polymers, penetration enhancers, emollients, skin irritation reducing agents, buffering agents, pH stabilizers, solubilizers, suspending agents, dispersing agents, stabilizers, plasticizers, tackifiers, surfactants, volatile chemicals, antioxidants, oxidants, chelating agents, complexing agents, diluents, excipients, material to prepare patch, material to prepare matrix patch, material to prepare reservoir patch etc.

Active agents may be dissolved, suspended, dispersed or uniformly mixed in the above stated single carrier, mixture of carriers and combinations of carrier. Any combination of two or more drugs such as such as tetrahydrocannabinol (THC), cannabidiol (CBD), or combinations thereof, and derivatives of these compounds may be dissolved, suspended, dispersed or uniformly mixed in the above stated single carrier, mixture of carriers and combinations of carrier.

The desired optimum transdermal and/or topical formulation of such as tetrahydrocannabinol (THC), cannabidiol (CBD), or combinations thereof, and derivatives of these compounds alone or in combinations thereof may comprise without any limitation to following carriers as stated from example 1 to example 12 either alone or in combinations thereof.

According to certain embodiments, transdermal compositions described herein are for the treatment and/or prevention and/or control of cancer, such as glioblastoma, GBM.

### Packaging/Treatment Kits

The disclosure provides a kit for conveniently and effectively carrying out the methods in accordance with the present disclosure. Such kits may be suited for the delivery of solid oral forms such as tablets or capsules. Such a kit may include a number of unit dosages. Such kits can include a means for containing the dosages oriented in the order of their intended use. An example of a means for containing the dosages in the order of their intended uses is a card. An example of such a kit is a “blister pack”. Blister packs are well known in the packaging industry and are widely used for packaging unit dosage forms. If desired, the blister can be in the form of a childproof blister, i.e., a blister that is difficult for a child to open, yet can be readily opened by an adult. If desired, a memory aid can be provided, for example in the form of numbers, letters, or other markings or with a calendar feature and/or calendar insert, designating the days and the sections of a day in the treatment schedule in which the dosages can be administered, such as, for example, an AM dose is packaged with a “midday” and a PM dose.; or an AM dose is packaged with a PM dose. Alternatively, placebo dosages, or vitamin or dietary supplements, either in a form similar to or distinct from the active dosages, can be included.

The disclosure provides compositions, including preparations, formulations and/or kits, comprising combinations of ingredients, as described above (including the multi-ingredient combinations of drugs of the invention), that are serviceable as therapies for treating, preventing or improving conditions, states and disease as provided in the invention. In one aspect, each member of the combination of ingredients is manufactured in a separate package, kit or container; or, all or a subset of the combinations of ingredients are manufactured in a separate package or container. In alternative aspects, the package, kit or container comprises a blister package, a clamshell, a tray, a shrink wrap and the like.

In one aspect, the package, kit or container comprises a “blister package” (also called a blister pack, or bubble pack). In one aspect, the blister package consists two or more separate compartments. This blister package is made up of two separate material elements: a transparent plastic cavity shaped to the product and its blister board backing. These two elements are then joined together with a heat sealing process which allows the product to be hung or displayed. Exemplary types of “blister packages” include: Face seal blister packages, gang run blister packages, mock blister packages, interactive blister packages, slide blister packages.

Blister packs, clamshells or trays are forms of packaging used for goods; thus, the invention provides for blister packs, clamshells or trays comprising a composition (e.g., a (the multi-ingredient combination of drugs of the invention) combination of active ingredients) of the invention. Blister packs, clamshells or trays can be designed to be non-reclosable, so consumers can tell if a package has already opened. They are used to package for sale goods where product tampering is a consideration, such as the agents of the invention. In one aspect, a blister pack of the invention comprises a moulded PVC base, with raised areas (the “blisters”) to contain the tablets, pills, etc. comprising the combinations of the invention, covered by a foil laminate. Tablets, pills, etc. are removed from the pack either by peeling the foil back or by pushing the blister to force the tablet to break the foil. In one aspect, a specialized form of a blister pack is a strip pack.

In one aspect, a blister pack also comprises a method of packaging where the compositions comprising combinations of ingredients of the disclosure are contained in-between a card and clear PVC. The PVC can be transparent so the item (patch, pill, tablet, geltab, etc.) can be seen and examined easily; and in one aspect, can be vacuum-formed around a mould so it can contain the item snugly and have room to be opened upon purchase. In one aspect, the card is brightly colored and designed depending on the item (patch, pill, tablet, geltab, etc.) inside, and the PVC is affixed to the card using pre-formed tabs where the adhesive is placed. The adhesive can be strong enough so that the pack may hang on a peg, but weak enough so that this way one can tear open the join and access the item. Sometimes with large items or multiple enclosed patches, pills, tablets, geltabs, etc., the card has a perforated window for access. In one aspect, more secure blister packs, e.g., for items such as pills, tablets, geltabs, etc. of the invention are used, and they can comprise of two vacuum-formed PVC sheets meshed together at the edges, with the informative card inside.

In one aspect, blister packaging comprises at least two components (e.g., is a multi-ingredient combination of drugs of the invention): a thermoformed “blister” which houses the product (e.g., a combination of the invention), and then a “blister card” that is a printed card with an adhesive coating on the front surface. During the assembly process, the blister component, which is most commonly made out of PVC, is attached to the blister card using a blister machine. Conventional blister packs can also be sealed.

As discussed herein, the products of manufacture of the invention can comprise the packaging of the therapeutic drug combinations of the invention, alone or in combination, as “blister packages” or as a plurality of packettes, including as lidded blister packages, lidded blister or blister card or packets, or a shrink wrap.

In one aspect, any of the invention's products of manufacture, including kits or blister packs, include memory aids to help remind patients when and how to take the agents of the invention.

The treatment kits can be constructed in a variety of forms familiar to one of ordinary skill in the art. The kits comprise at least one unit dosage of an active for administration according to a daily regimen and a means for containing the unit dosages. The treatment kits can, for example, be constructed for administration once daily, twice daily, thrice daily, four times daily, multiple administrations daily, or other dosage regimens. The kits comprise a means for the daily administration of an agent of the invention. In one embodiment the kits include from about one to about four unit dosages.

In one embodiment, the means for containing the unit dosages is a card, including, for example, a card that is capable of being folded. This card will be referred to herein as a main card, or alternatively a principal card or a first card, to distinguish it from additional optional cards, circulars, or other such materials which can be associated with the kit. This main card can be folded with a simple crease, or alternatively, with a double crease, so as to exhibit a spine, similar to the spine of a closed book. The main card can comprise a printable surface, i.e. a surface upon which the product name, appropriate administration instructions, product information, drawings, logos, memory aids, calendar features, etc. can be printed. The main card can comprise a means for containing said unit dosage or different dosages designated for different time of the day, and a memory aid for administering said unit dosage or dosages. The main card, especially if it is prepared from two or more laminated paperboard surfaces, can comprise a slit or pocket, for example in one of the inner paperboard surfaces of the folded card. The slit or pocket can be used to contain a removable secondary card, i.e., a second card or insert card, which is not permanently attached or affixed to the main card.

The memory aid can include a listing of the days of the week, i.e., Sunday, Monday, Tuesday, Wednesday, Thursday, Friday, and Saturday, with appropriate spaces for the patient to select and indicate on the card the preferred day of the week on which to administer the therapy. The memory aid can include a listing of the time of day with appropriate spaces for the patient to select and indicate on the card the preferred time of day (e.g.: AM, PM, midday) at which to administer the therapy. The memory aid can also include removable stickers having an appropriate pressure sensitive adhesive to facilitate easy removal and refastening to a desired surface such as a calendar or dayminder. The removable stickers can be located on the main card, or can be located on the secondary card which is constructed so that it can be readily inserted into and removed from the optional slit in the main card. Additionally, the optional slit can contain additional patient information and other circulars.

Other means for containing said unit dosages can include bottles and vials, wherein the bottle or vial comprises a memory aid, such as a printed label for administering said unit dosage or dosages. The label can also contain removable reminder stickers for placement on a calendar or dayminder to further help the patient to remember when to take a dosage or when a dosage has been taken.

The invention will be illustrated in more detail with reference to the following Examples, but it should be understood that the present invention is not deemed to be limited thereto.

### EXAMPLES

### Example 1

The transdermal formulation and/or topical formulation of the disclosure may comprise solvents known to those skilled in the art either alone or in combinations thereof without any limitation to following like alcohol C₁-C₂₀ such as but not limited to (methanol, ethanol, isopropyl alcohol, butanol, propanol etc.), polyhydric alcohols, glycols such as but not limited to (propylene glycol, polyethylene glycol, dipropylene glycol, hexylene glycol, butylene glycol, glycerine etc.), derivative of glycols, pyrrolidone such as but not limited to (N methyl 2-pyrrolidone, 2-pyrrolidone etc.), sulfoxides such as but not limited to (dimethyl sulfoxide, decylmethylsulfoxide etc.), dimethylisosorbide, mineral oils, vegetable oils, water, polar solvents, semi polar solvents, non polar solvents, volatile chemicals which can be used to make matrix patch such as but not limited to (ethanol, propanol, ethyl acetate, acetone, methanol, dichloromethane, chloroform, toluene, IPA), acids such as but not limited to acetic acid, lactic acid, levulinic acid, bases and others. More preferably in the range of 0.01%-95% w/w or w/v. In exemplary embodiments, formulations of the disclosure may comprise solvents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solvents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solvents will represent approximately 1 wt % to 75 wt %, preferably 2 wt % to 30 wt %, more preferably 5 wt. % to 20 wt. % of the formulation.

### Example 2

The transdermal formulation and/or topical formulation of the disclosure may comprise gelling agents and/or thickening and/or suspending agents known to those skilled in the art either alone or in combinations thereof without any limitation to following like natural polymers, polysaccharides and its derivatives such as but not limited to (agar, alginic acid and derivatives, cassia tora, collagen, gelatin, gellum gum, guar gum, pectin, potassium, or sodium carageenan, tragacanth, xantham, gum copal, chitosan, resin etc.), semisynthetic polymers and its derivatives such as without any limitation to cellulose and its derivatives (methylcellulose, ethyl cellulose, carboxymethyl cellulose, hydroxylpropyl cellulose, hydroxylpropylmethyl cellulose etc.), synthetic polymers and its derivatives such as without any limitation to carboxyvinyl polymers or carbomers (carbopol 940, carbopol 934, carbopol 971p NF), polyethylene, and its copolymers etc., clays such as but not limited to (silicates, bentonite), silicon dioxide, polyvinyl alcohol, acrylic polymers (eudragit), acrylic acid esters, polyacrylate copolymers, polyacrylamide, polyvinyl pyrrolidone homopolymer and polyvinyl pyrrolidone copolymers such as but not limited to (PVP, Kollidon 30, poloxamer), isobutylene, ethyl vinyl acetate copolymers, natural rubber, synthetic rubber, pressure sensitive adhesives such as silicone polymers such as but not limited to (bio psa 4302, bio-psa 4202 etc.), acrylic pressure sensitive adhesives such as but not limited to (duro-tak 87-2156, duro-tak 387-2287, etc.), polyisobutylene such as but not limited to (polyisobutylene low molecular weight, plyisobutylene medium molecular weight, polyisobutylene 35000 mw, etc.), acrylic copolymers, rubber based adhesives, hot melt adhesives, styrene-butadiene copolymers, bentonite, all water and/or organic solvent swellable polymers, etc. In exemplary embodiments, formulations of the disclosure may comprise gelling agents and/or thickening and/or suspending agents at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise gelling agents and/or thickening and/or suspending agents at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the gelling agents and/or thickening and/or suspending agents will represent approximately 1 wt % to 75 wt %, preferably 2 wt % to 30 wt %, more preferably 5 wt. % to 20 wt. % of the formulation. More preferably in the range of 0.1% 70% w/w or w/v.

### Example 3

The transdermal formulation and/or topical formulation of the disclosure may comprise permeation enhancers known to those skilled in the art either alone or in combination thereof without any limitation to the following, such as sulfoxides, and similar chemicals such as but not limited to (dimethylsulfoxide, dimethylacetamide, dimethylformamide, decymethylsulfoxide, dimethylisosorbide etc.), azone, pyrrolidones such as but not limited to (N-methyl-2-pyrrolidone, 2-pyrrolidon etc.), esters, fatty acid esters such as but not limited to (propylene glycol monolaurate, butyl ethanoate, ethyl ethanoate, isopropyl myristate, isopropyl palmitate, methyl ethanoate, decyl oleate, glycerol monooleate, glycerol monolaurate, lauryl laurate etc.), fatty acids such as but not limited to (capric acid, caprylic acid, lauric acid, oleic acid, myristic acid, linoleic acid, stearic acid, palmitic acid etc.), alcohols, fatty alcohols and glycols such as but not limited to (oleyl alcohol, nathanol, dodecanol, propylene glycol, glycerol etc.), ethers alcohol such as but not limited to (diethylene glycol monoethyl ether), urea, triglycerides such as but not limited to triacetin, polyoxyethylene fatty alcohol ethers, polyoxyethylene fatty acid esters, esters of fatty alcohols, essential oils, surfactant type enhancers such as but not limited to (brij, sodium lauryl sulfate, tween, polysorbate), terpene, terpenoids and all penetration or permeation enhancers referred in the book “Percutaneous Penetration Enhancers” (Eric W. Smith, Howard I. Maibach, 2005. November, CRC press). In exemplary embodiments, formulations of the disclosure may comprise permeation enhancers at a concentration of about 0.010%, about 0.02%, about 0.05%, about 0.10%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise permeation enhancers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the permeation enhancers will represent approximately 1 wt % to 75 wt %, preferably 2 wt % to 30 wt %, more preferably 5 wt. % to 20 wt. % of the formulation. More preferably in the range of 0.01%-95% w/w or w/v.

### Example 4

The transdermal formulation and/or topical formulation of the disclosure may comprise plasticizers known to those skilled in the art either alone or in combination thereof without any limitation to following like glycerol and its esters, phosphate esters, glycol derivatives, sugar alcohols, sebacic acid esters, citric acid esters, tartaric acid esters, adipate, phthalic acid esters, triacetin, oleic acid esters and all the plasticizers which can be used in transdermal drug delivery system referred in the book “Handbook of Plasticizers” (George Wypych, 2004, Chem Tec Publishing). In exemplary embodiments, formulations of the disclosure may comprise plasticizers at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise plasticizers at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the plasticizers will represent approximately 1 wt % to 75 wt %, preferably 2 wt % to 30 wt 00 more preferably 5 wt. % to 20 wt. % of the formulation. More preferably in the range of 0.01%-95% w/w or w/v.

### Example 5

The transdermal formulation and/or topical formulation of the disclosure may comprise emollients, humectants, skin irritation reducing agents and similar compounds or chemicals known to those skilled in the art either alone or in combinations thereof without any limitation to following like petrolatum, lanolin, mineral oil, dimethicone, zinc oxide, glycerin, propylene glycol and others. More preferably in the range of 0.01%-95% w/w or w/v. In exemplary embodiments, formulations of the disclosure may comprise emollients, humectants, skin irritation reducing agents and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise emollients, humectants, skin irritation reducing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the emollients, humectants, skin irritation reducing agents and similar compounds will represent approximately 1 wt % to 75 wt %, preferably 2 wt % to 30 wt %, more preferably 5 wt. % to 20 wt. % of the formulation. More preferably in the range of 0.01%-95% w/w or w/v.

### Example 6

The transdermal formulation and/or topical formulation of the disclosure may comprise solubilizers, surfactants, emulsifying agents, dispersing agents and similar compounds or chemicals known to those skilled in the art either alone or in combination thereof without any limitation to following like polysorbate such as but not limited to (polysorbate 20, polysorbate 40, polysorbate 60, polysorbate 80 etc.), span such as but not limited to (span 80, span 20 etc.), surfactants such as (anionic, cationic, nonionic and amphoteric), propylene glycol monocaprylate type I, propylene glycol monocaprylate type II, propylene glycol dicaprylate, medium chain triglycerides, propylene glycol monolaurate type II, linoleoyl polyoxyl-6 glycerides, oleoyl-polyoxyl-6-glycerides, lauroyl polyoxyl-6-gylcerides, polyglyceryl-3-dioleate, diethylene glycol monoethyl ether, propylene glycol monolaurate type I, polyglyceryl-3-dioleate, caprylocaproyl polyoxyl—8 glycerides etc., cyclodextrins and others. More preferably in the range of 0.010% 95% w/w or w/v. In exemplary embodiments, formulations of the disclosure may comprise solubilizers, surfactants, emulsifying agents, dispersing agents and similar compounds at a concentration of about 0.010%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 210%, about 22%, about 23%, about 24%, about 25%, about 26%, about 27%, about 28%, about 29%, about 30%, about 35%, about 40%, about 45%, about 50%, about 55%, about 60%, about 61%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise solubilizers, surfactants, emulsifying agents, dispersing agents and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the solubilizers, surfactants, emulsifying agents, dispersing agents and similar compounds will represent approximately 1 wt % to 75 wt %, preferably 2 wt % to 30 wt %, more preferably 5 wt. % to 20 wt. % of the formulation. More preferably in the range of 0.01% 95% w/w or w/v.

### Example 7

Different techniques and ingredients can be used to increase the stability and/or solubility of the active agents in formulation such as without any limitation to coating, encapsulation, microencapsulation, nanoencapsulation, lyophilization, chelating agents, complexing agents, etc.

### Example 8

The transdermal formulation and/or topical formulation of the disclosure may comprise auxiliary pH buffering agents and pH stabilizers and similar compounds known to those skilled in the art which helps to maintain the appropriate pH of formulation preferably in the range of 4.0-8.0 either alone or in combination thereof without any limitation to following such as phosphate buffer, acetate buffer, citrate buffer, etc., acids such as but not limited to (carboxylic acids, inorganic acids, sulfonic acids, vinylogous carboxylic acids and others), base such as but not limited to (sodium hydroxide, potassium hydroxide, ammonium hydroxide, triethylamine, sodium carbonate, sodium bicarbonate) etc. More preferably in the range of 0.01%-30% w/w or w/v. In exemplary embodiments, formulations of the disclosure may comprise pH buffering agents and pH stabilizers and similar compounds at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 21%, about 22%, about 23%, about 24%, about 25%, about 26%, about 27%, about 28%, about 29%, about 30%, about 35%, about 40%, about 45%, about 50%, about 55%, about 60%, about 610%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise pH buffering agents and pH stabilizers and similar compounds at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the pH buffering agents and pH stabilizers and similar compounds will represent approximately 1 wt % to 75 wt %, preferably 2 wt % to 30 wt %, more preferably 5 wt. % to 20 wt. % of the formulation. More preferably in the range of 0.01%-30% w/w or w/v.

### Example 9

The transdermal formulation and/or topical formulation of the disclosure may comprise antioxidants such as but not limited to (sodium metabisulfite, citric acid, ascorbic acid, BHA, BHT), oxidizing agents, stabilizers, discoloring agents, preservatives and similar compounds or chemicals known to those skilled in the art which helps to get a stable formulation can be used either alone or in combination thereof without any limitation. More preferably in the range of 0.01%-50% w/w or w/v. In exemplary embodiments, formulations of the disclosure may comprise antioxidants at a concentration of about 0.01%, about 0.02%, about 0.05%, about 0.1%, about 0.2%, about 0.3%, about 0.4%, about 0.5%, about 0.6%, about 0.7%, about 0.8%, about 0.9%, about 1%, about 2%, about 3%, about 4%, about 5%, about 6%, about 7%, about 8%, about 9%, about 10%, about 11%, about 12%, about 13%, about 14%, about 15%, about 16%, about 17%, about 18%, about 19%, about 20%, about 210%, about 22%, about 23%, about 24%, about 25%, about 26%, about 27%, about 28%, about 29%, about 30%, about 35%, about 40%, about 45%, about 50%, about 55%, about 60%, about 610%, about 62%, about 63%, about 64%, about 65%, about 66%, about 67%, about 68%, about 69%, about 70%, about 75%, about 75%, and about 80% of the formulation. In exemplary embodiments, formulations of the disclosure may comprise antioxidants at a concentration of about 1 to 20%, of about 5% to 25%, about 10% to about 20%, or about 15% to about 18%, about 30% to about 70%, about 35% to about 65%, about 63.13%, and about 40% to about 64% w/w. In exemplary formulations of the disclosure, the antioxidants will represent approximately 1 wt % to 75 wt %, preferably 2 wt % to 30 wt %, more preferably 5 wt. % to 20 wt. % of the formulation. More preferably in the range of 0.01%-50% w/w or w/v.

### Example 10

The transdermal formulation and/or topical formulation of the disclosure may be formulated in ointment and/or cream base known to those skilled in the art.

### Example 11

Materials to make the continuous drug delivery systems and methods of the disclosure is known to those skilled in the art, for example, such as but not limited to a pump, transdermal patch, topical patch, reservoir patch, matrix patch, drug in adhesives, film forming formulation, micro-dosing transdermal patch, transdermal films and may include, such as but are not limited to polymers, copolymers, derivatives, backing film, release membranes, release liners, etc. either alone or in combinations thereof. Pressure sensitive adhesives (such as but not limited to silicone polymers, rubber based adhesives, acrylic polymers, acrylic copolymers, polyisobutylene, acrylic acid—isooctyl acrylate copolymer, hot melt adhesives, polybutylene etc.), backing film (such as but not limited to ethylene vinyl acetate copolymers, vinyl acetate resins, polyurethane, polyvinyl chloride, metal foils, polyester, aluminized films, polyethylene, etc.), release membrane (such as but not limited to microporous polyethylene membrane, microporous polypropylene membrane, rate controlling ethylene vinyl acetate copolymer membrane etc.), release liners (such as but not limited to siliconized polyester films, fluoropolymer coated polyester film, polyester film, siliconized polyethylene terephthalate film, etc.), tapes, etc.

The continuous and/or transdermal formulation and/or topical formulation and/or transdermal delivery system of the disclosure may deliver at least therapeutic effective dose of active agent, such as for example, tetrahydrocannabinol (THC), cannabidiol (CBD), or combinations thereof, and derivatives of these compounds, alone or in combinations thereof in human plasma required for treatment and/or prevention and/or control of cancer, such as glioblastoma, GBM. Therapeutically effective active agent such as tetrahydrocannabinol (THC), cannabidiol (CBD), or combinations thereof, and derivatives of these compounds dosages refers to the therapeutic concentration of in human plasma required for treating and/or preventing treatment and/or prevention and/or control of cancer, such as glioblastoma, GBM. Furthermore, the precise therapeutic effective dose of such as tetrahydrocannabinol (THC), cannabidiol (CBD), or combinations thereof, and derivatives of these compounds in the transdermal formulation or topical formulation or transdermal delivery system can be determined by those skilled in the art based on factors such as but not limited to the patient's condition etc. The transdermal formulation or topical formulation or transdermal delivery system will be available in different dosage strengths and patch sizes in order to achieve optimum therapeutic outcome based on patient's requirement.

In yet another embodiment, the transdermal formulation and/or topical formulation and/or transdermal delivery system of the disclosure may deliver at least therapeutic effective dose of such as tetrahydrocannabinol (THC), cannabidiol (CBD), or combinations thereof, and derivatives of these compounds. Therapeutically effective such as tetrahydrocannabinol (THC), cannabidiol (CBD), or combinations thereof, and derivatives of these compounds refers to the therapeutic concentration of highly purified thereof in human plasma required for the treatment and/or prevention and/or control of cancer, such as glioblastoma, GBM.

The transdermal formulation or transdermal patch of active agents such as tetrahydrocannabinol (THC), cannabidiol (CBD), or combinations thereof, and derivatives of these compounds can be applied to the skin surface in any of the following dosage regimens such as once in a day, once in two days, once in three days, once in four days, once in five days, once in six days, once in a week, once in a range of from about 8 to about 13 days, once in two weeks, or once in 15 days.

### Example 12

Synthetic cannabidiol (CBD) formulations for transdermal delivery ((Formulation Nos. 047-055) were prepared by mixing ingredients as shown in Table 1:

TABLE 1
Transdermal Synthetic Cannabidiol formulations

| Excipients | CBD 047 | CBD 048 | CBD 049 | CBD 050 | CBD 051 | CBD 052 | CBD 053 | CBD 054 | CBD 055 |
|---|---|---|---|---|---|---|---|---|---|
| CBD | 4.84% | 4.98% | 4.73% | 4.99% | 4.87% | 4.89% | 5.04% | 4.83% | 5.00% |
| DURO-TAK 2516 | 95.16% | — | — | — | — | — | — | — | — |
| DURO-TAK | — | 95.02% | — | — | — | — | — | — | — |
| 9301 | | | | | | | | | |
| DURO-TAK | — | — | 95.27% | — | — | — | — | — | — |
| 2287 | | | | | | | | | |
| DURO-TAK | — | — | — | 95.01% | — | — | — | — | — |
| 2054 | | | | | | | | | |
| DURO-TAK 2852 | — | — | — | — | 95.13% | — | — | — | — |
| DURO-TAK 2074 | — | — | — | — | — | 95.11% | — | — | — |
| DURO-TAK | — | — | — | — | — | — | 94.96% | — | — |
| 2194 | | | | | | | | | |
| BIO-PSA 4501 | — | — | — | — | — | — | — | 95.17% | — |
| B10-PSA 4201 | — | — | — | — | — | — | — | — | 95.00% |

The above ingredients (Table 1) are blended by stirring for 18 hours and then, using a commercial benchtop spreader, the matrix is evenly spread onto an 8×14 inch sheet of release liner (such as 3M 9744) to a thickness of 0.5 mm. The sheet is then place in an oven at 86 F for 120 min to evaporate off the ethyl acetate adhesive solvent. An opaque backing membrane (such as 3M 9730 NR film) with low permeability to oxygen to inhibit photo and oxidative degradation, is then carefully applied by hand to avoid formation of bubbles and voids. A circular die (1.5 inches diameter) is used to cut patches (1.76 sqcm) for subsequent studies. After drying, the drug adhesive matrix has a surface density of 2-30 mg/sqcm, containing CBD in 5% w/w.

The prepared formulations where then subjected to a release study as follows: After weighing the patches (n=3), the release liner was removed, and the patches were placed in 20 ml scintillation vials with 15 ml of receiving media. The receiving media was PBS solution of pH 7.4 with 0.5% Brij(O)20. Vials were placed on the roller overnight at 20 RPM. Samples were withdrawn every 24 hours, up to 72 hours, and media was fully replaced each time. Samples were then run in the HPLC in order to determine the 0% release of CBD from the different formulations.

The prepared formulations also analyze for the uniformity of drug content. The patches (n=3) were weight out for each formulation, the release liner was removed, and the patches (including the release liner) were placed in 20 ml scintillation vials with 15 ml of solution IPA:Ethanol (190proof) (50:50). The vials were then placed on the roller at 20 RPM and left overnight. Samples were withdrawn from each vial and analyzed on the HPLC to determine the drug content of each formulation.

TABLE 2
Experimental Condition for In-vitro Permeability testing

| | PBS (pH 7.4) + 0.5% Brij-O(20) + |
|---|---|
| Receiving Media | 0.01% Sodium Azide |
| Receiving Media Volume (mL) | 15 |
| Sample Volume (mL) | 15 |
| Sampling Interval (hr) | 24, 48 |
| Diffusion area (sqcm) | 1.76 |

% Release of CBD through the matrix system was measured for a minimum period of 48 Hrs (2 days) and results of the 00 release are provided in Table 3.

TABLE 3
% CBD Release Results

| | CBD 047 | CBD 048 | CBD 049 | CBD 050 | CBD 051 | CBD 052 | CBD 053 | CBD 054 | CBD 055 |
|---|---|---|---|---|---|---|---|---|---|
| Av. % | 26 (3) | 9 (7) | 20 (7) | 40 (2) | 23 (8) | 35 (12) | 37 (6) | 93 (2) | Did not |
| Release at 24 | | | | | | | | | solubilize |
| hours | | | | | | | | | the CBD |
| Av. % Release | 18 (7) | 6 (12) | 13 (13) | 22 (1) | 13 (9) | 12 (1) | 22 (5) | 2 (2) | 98% |
| at 48 hours | | | | | | | | | (0.05) |
| % Release | 44 | 15 | 33 | 62 | 36 | 47 | 59 | 95 | |
| 0-48hours | | | | | | | | | |
| (cumulative) | | | | | | | | | |
| % CBD from | 104 (2) | 100 (1) | 100 (5) | 102 (1) | 103 (6) | 96 (15) | 104 (4) | 107 | |
| extraction | | | | | | | | | |
| (STD) | | | | | | | | | |

Drug content study showed that the 00 recover of CBD in extraction is between 96-10700 for all the manufactured formulations. Furthermore, the release study showed that the silicone adhesive 4501 showed more than 90% release within first 24 hrs. Based on the release profile following are the best adhesive for CBD formulation: BIOPSA-4501>2054=2194>2074>2516>2852=2287>9301.

Release studies indicate that the functional group and crosslinker affect the CBD release from acrylic adhesive. According to current study, acrylic adhesive containing —COOH functional group with crosslinker showed the maximum release of CBD from all the acrylic adhesive patches.

### Example 13

Additional synthetic Cannabidiol (CBD) formulations for transdermal delivery (Formulation Nos. 057 through 064) were prepared by mixing ingredients as shown in Table 4:

TABLE 4
Transdermal Synthetic Cannabidiol formulation no. 057 to 064

| Excipients | CBD 057 | CBD 058 | CBD 059 | CBD 060 | CBD 061 | CBD 062 | CBD 063 | CBD 064 |
|---|---|---|---|---|---|---|---|---|
| CBD | 5.0% | 5.0% | 5.0% | 4.9% | 4.8% | 4.8% | 4.9% | 4.8% |
| BIO-PSA 4501 | 95.0% | 84.6% | 84.5% | 85.0% | 83.3% | 89.7% | 86.9% | 89.6% |
| IPM | — | 10.4% | — | — | — | — | — | — |
| IPP | — | — | 10.5% | — | — | — | — | — |
| Oleic Acid | — | — | — | 10.1% | — | — | — | — |
| Transcutol P | — | — | — | — | 11 .9% | — | — | — |
| Brij O20 | — | — | — | — | — | 5.5% | — | — |
| Poloxamer 124 | — | — | — | — | — | — | 8.2% | — |
| PGML | — | — | — | — | — | — | — | 5.6% |

Synthetic Cannabidiol formulations for transdermal delivery (057-064) were prepared by the same procedure described in Example 12.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD formulations prepared as described above. The receptor compartment was filled with receptor medium, held at constant temperature, and constantly stirred to collect the CBD as it diffuses through the skin and into receptor compartment. It is important to confirm that the receptor fluid is always in contact with the skin. The receptor compartment was emptied at 24 hr intervals for assay of CBD and replaced with fresh receptor solution. In order to maintain the sink condition in receptor compartment, it is important to keep the CBD concentration in receptor compartment less than 10% of its solubility. The experimental conditions are provided in Table 5:

TABLE 5
Experimental Condition for In-vitro Permeability testing

| | De-ionized water + 0.5% Brij-O(20) + |
|---|---|
| Receiving Media | 0.01% Sodium Azide |
| Receiving Media Volume (mL) | 13 |
| Sample Volume (mL) | 13 |
| Sampling Interval (hr) | 24, 48, 72, 96 |
| Franz-cell diffusion area (sqcm) | 1.76 |
| Membrane Type | Human Cadaver Skin |
| Mixing Speed | 600 RPM |

Flux of CBD through the human cadaver skin was measured for a minimum period of 96 Hrs (4 days) and results of the flux measurement are provided in Table 6.

Upon completion of the flux study, the used patches were carefully removed and extract the CBD from the use patches using IPA:Ethanol (50:50). The human cadaver skin was also soaked in IPA:Ethanol (50:50), in order to extract the CBD from it. The samples were analyzed using HPLC. The data in table 6 showed the amount of CBD present in the skin and the left-over patches.

TABLE 6
CBD Flux Results

| | CBD 057 | CBD 058 | CBD 059 | CBD 060 | CBD 061 | CBD 062 | CBD 063 | CBD 064 |
|---|---|---|---|---|---|---|---|---|
| Av. cumulative | 52 | 31 | 16 | 120 | 86 | BLLQ | BLLQ | 53 |
| amount permeated | | | | | | | | |
| at 96 hrs (μg) | | | | | | | | |
| Av. flux 24- | 0.41 | 0.37 | 0.37 | 0.69 | 0.51 | BLLQ | BLLQ | 0.42 |
| 96 hrs (μg/hr/sqcm) | | | | | | | | |
| Peak flux | 0.44 | 0.38 | 0.38 | 0.82 | 0.60 | BLLQ | BLLQ | 0.44 |
| (μg/hr/sqcm) | | | | | | | | |
| Time to peak flux | 72 | 72 | 72 | 72 | 72 | 0 | 0 | 72 |
| (hrs) | | | | | | | | |

| | | | | | | | |
|---|---|---|---|---|---|---|---|
| Av. CBD Amount in | 1.57 | Did not Determined | 1.14 | 2.26 | 1.81 | 1.88 | 1.56 |
| patch (mg) | | | | | | | |
| Av. CBD Amount in | 0.01 | | 0.59 | 0.13 | 0.54 | 0.24 | 0.10 |
| skin (mg) | | | | | | | |

### Example 14

Additional synthetic Cannabidiol (CBD) formulations for transdermal delivery (Formulation Nos. 102 through 106) were prepared by mixing ingredients as shown in Table 7:

TABLE 7
Transdermal Synthetic Cannabidiol formulation no. 102 to 106

| Excipients | CBD-102 | CBD-103 | CBD-104 | CBD-105 | CBD-106 |
|---|---|---|---|---|---|
| CBD | 3.69% | 4.01% | 3.90% | 3.97% | 3.94% |
| Oleic acid | 3.69% | — | 4.30% | — | 9.06% |
| Propylene | 12.19% | 13.22% | 12.50% | 13.49% | — |
| Glycol | | | | | |
| Isopropyl | — | 4.41% | 4.69% | — | 6.31% |
| Palmitate | | | | | |
| BIO-PSA 4501 | 80.42% | 78.37% | 74.62% | 82.54% | 80.69% |

Synthetic Cannabidiol formulations for transdermal delivery (102-106) were prepared by the same procedure described in Example 12.

The in-vitro flux study was performed by the same procedure described in Example 13. Flux of CBD through the human cadaver skin was measured for a minimum period of 144 Hrs (6 days) and results of the flux measurement are provided in Table 8.

TABLE 8
CBD Flux Results

| Excipients | CBD-102 | CBD-103 | CBD-104 | CBD-105 | CBD-106 |
|---|---|---|---|---|---|
| Avg Flux 0- | 0.23 | 0.51 | 0.12 | 0.13 | 0.00 |
| 24 hr, | (0.37) | (87.37) | (173.21) | (173.21) | |
| μg/sqcm/hr | | | | | |
| (% RSD) | | | | | |
| Avg Flux 24- | 0.44 | 0.30 | 0.32 | 0.31 | 0.30 |
| 90 hr, | (8.00) | (2.80) | (14.52) | (12.19) | (22.80) |
| μg/sqcm/hr | | | | | |
| (% RSD) | | | | | |
| Avg Flux 90- | 0.53 | 0.40 | 0.41 | 0.35 | 0.36 |
| 120 hr, | (10.96) | (13.95) | (13.50) | (13.20) | (16.54) |
| μg/sqcm/hr | | | | | |
| (% RSD) | | | | | |
| Avg Flux 120- | 0.51 | 0.42 | 0.44 | 0.27 | 0.27 |
| 144 hr, | (9.02) | (11.41) | (8.42) | (15.04) | (87.40) |
| μg/sqcm/hr | | | | | |
| (% RSD) | | | | | |
| Avg Flux 24- | 0.43 | 0.41 | 0.32 | 0.26 | 0.23 |
| 144 hr, | (7.09) | (28.88) | (12.14) | (13.47) | (31.69) |
| μg/sqcm/hr | | | | | |
| (% RSD) | | | | | |

### Example 15

Additional synthetic Cannabidiol (CBD) formulations for transdermal delivery (Formulation No. 060) were prepared by mixing ingredients as shown in Table 9:

TABLE 9
Transdermal Synthetic Cannabidiol formulation no. 60

| Excipients | CBD-060 |
|---|---|
| CBD | 4.84% |
| Oleic acid | 10.01% |
| BIO-PSA 4501 | 85.15% |

Synthetic Cannabidiol formulations for transdermal delivery (060) were prepared by the same procedure described in Example 12.

The in-vitro flux study was performed by the same procedure described in Example 13. The effect of receiving media (RM) was evaluated using several different RM: 1) 0.50% w/w Brij O20 in water, 2) 1% w/w Brij O20 in water, 3) 0.5% w/w Brij O20 in water:Ethanol (50:50), 4) 0.5% w/w Brij O20 in Water:Ethanol (90:10), 5) 0.5% w/w Brij O20 in Water:Ethanol (80:20), 6) 0.5% w/w Brij O20 in Water:Ethanol (70:30) and, 7) 0.5% w/w Brij O20 in Water:PEG400 (60:40). Flux of CBD through the human cadaver skin was measured for a minimum period of 168 Hrs (7 days) and results of the flux measurement are provided in Table 10, Table 11 and Table 12.

TABLE 10
CBD Flux Results

| | DI water +30 | DI water +30 | DI water with 0.5% |
|---|---|---|---|
| | 0.5% Brij | 1.0% Brij | Brij O20 (50%) + |
| Receiving Media | O20 | O20 | ethanol (50%) |
| Avg Flux 0- | BLLQ | BLLQ | 6.81 (12.65) |
| 24 hr, μg/sqcm/hr | | | |
| (% RSD) | | | |
| Avg Flux 24- | BLLQ | BLLQ | 5.01 (10.46) |
| 48 hr, μg/sqcm/hr | | | |
| (% RSD) | | | |
| Avg Flux 48- | BLLQ | BLLQ | 3.73 (21.04) |
| 72 hr, μg/sqcm/hr | | | |
| (% RSD) | | | |
| Avg Flux 72- | BLLQ | BLLQ | 1.24 (21.31) |
| 96 hr, μg/sqcm/hr | | | |
| (% RSD) | | | |
| Avg Flux 96- | BLLQ | BLLQ | 4.14 (22.31) |
| 120 hr, μg/sqcm/hr | | | |
| (% RSD) | | | |
| Avg Flux 120- | BLLQ | BLLQ | 3.08 (31.94) |
| 144 hr, μg/sqcm/hr | | | |
| (% RSD) | | | |
| Avg Flux 144- | BLLQ | BLLQ | 0.55 (81.13) |
| 168 hr, μg/sqcm/hr | | | |
| (% RSD) | | | |

TABLE 11
CBD Flux Results

| | DI water with | DI water with | DI water with |
|---|---|---|---|
| | 0.5% Brij O20 | 0.5% Brij O20 | 0.5% Brij O20 |
| | (90%) + | (80%) + | (50%) + |
| Receiving Media | ethanol (10%) | ethanol (20%) | ethanol (50%) |
| Avg Flux 0- | BLLQ | 0.15 (154.97) | 5.58 (24.33) |
| 24 hr, μg/sqcm/hr | | | |
| (% RSD) | | | |
| Avg Flux 24- | 0.24 (111.0) | 0.13 (154.92) | 4.20 (11.02) |
| 48 hr, μg/sqcm/hr | | | |
| (% RSD) | | | |
| Avg Flux 48- | 0.15 (155.34) | 0.08 (244.95) | 5.16 (25.90) |
| 72 hr, μg/sqcm/hr | | | |
| (% RSD) | | | |
| Avg Flux 72- | 0.27 (77.80) | 0.08 (244.95) | 4.01 (18.19) |
| 96 hr, μg/sqcm/hr | | | |
| (% RSD) | | | |
| Avg Flux 96- | 0.49 (10.47) | 0.15 (155.68) | 3.37 (13.85) |
| 120 hr, μg/sqcm/hr | | | |
| (% RSD) | | | |
| Avg Flux 120- | 0.42 (49.57) | 0.16 (155.47) | 2.54 (29.27) |
| 144 hr, μg/sqcm/hr | | | |
| (% RSD) | | | |
| Avg Flux 144- | BLLQ | 0.18 (138.85) | 0.44 (99.0) |
| 168 hr, μg/sqcm/hr | | | |
| (% RSD) | | | |

TABLE 12
CBD Flux Results

| | DI water with | DI water with | DI water |
|---|---|---|---|
| | 0.5% Brij O20 | 0.5% Brij O20 | (60%) + |
| | (70%) + | (50%) + | PEG- |
| Receiving Media | ethanol (30%) | ethanol (50%) | 400 (40%) |
| Avg Flux 0- | 0.36 (79.23) | 4.49 (37.81) | BLLQ |
| 24 hr, μg/sqcm/hr | | | |
| (% RSD) | | | |
| Avg Flux 24- | 11.69 (8.97) | 0.49 (18.12) | 1.23 (31.26) |
| 48 hr, μg/sqcm/hr | | | |
| (% RSD) | | | |
| Avg Flux 48- | 1.68 (27.58) | 13.07 (84.62) | 0.62 (14.70) |
| 72 hr, μg/sqcm/hr | | | |
| (% RSD) | | | |
| Avg Flux 72- | 1.32 (22.94) | 5.80 (33.13) | 0.62 (13.38) |
| 96 hr, μg/sqcm/hr | | | |
| (% RSD) | | | |
| Avg Flux 96- | 0.98 (20.19) | 3.19 (42.69) | 0.66 (14.62) |
| 120 hr, μg/sqcm/hr | | | |
| (% RSD) | | | |

### Example 16

Additional synthetic Cannabidiol (CBD) formulations for transdermal delivery (Formulation No. 102 and 104) were prepared by mixing ingredients as shown in Table 13:

TABLE 13
Transdermal Synthetic Cannabidiol formulation no. 102 and 104

| Excipients | CBD-102 | CBD-104 |
|---|---|---|
| CBD | 4.34 | 4.21 |
| PG | 13.45 | 13.04 |
| Oleic Acid | 4.34 | 4.63 |
| IPP | — | 4.21 |
| BIO-PSA 4501 | 77.86 | 73.91 |

Synthetic Cannabidiol formulations for transdermal delivery (102 and 104) were prepared by the same procedure described in Example 12.

The in-vitro flux study was performed by the same procedure described in Example 13. The effect of receiving media (RM) was evaluated using two different RM: 1) 0.5% w/w Brij O20 in water, and 2) 0.5% w/w Brij O20 in water: Ethanol (50:50. Flux of CBD through the human cadaver skin was measured for a minimum period of 120-144 Hrs (5-6 days) and results of the flux measurement are provided in Table 14

TABLE 14
CBD Flux Results

| | Formulation | | | |
|---|---|---|---|---|
| | CBD-102 | | CBD-104 | |
| | | DI water with | | DI water with |
| | DI water + | 0.5% Brij | DI water + | 0.5% Brij |
| Receiving | 0.5% | O20 (50%) + | 0.5% | O20 (50%) + |
| Media | Brij O20 | ethanol (50%) | Brij O20 | ethanol (50%) |
| Avg Flux 0-24 | BLLQ | 4.10 (29.62) | BLLQ | 8.54 (9.22) |
| hr, μg/sqcm/hr | | | | |
| (% RSD) | | | | |
| Avg Flux 24-48 | BLLQ | 9.94 (1.99) | BLLQ | 9.67 (12.07) |
| hr,μg/sqcm/hr | | | | |
| (% RSD) | | | | |
| Avg Flux 48-72 | 0.52 (10.97) | 13.87 (1.61) | BLLQ | 5.62 (24.78) |
| hr, μg/sqcm/hr | | | | |
| (% RSD) | | | | |
| Avg Flux 72-96 | 0.42 (10.54) | 2.80 (4.14) | BLLQ | 3.66 (40.98) |
| hr, μg/sqcm/hr | | | | |
| (% RSD) | | | | |
| Avg Flux 96- | 0.40 (7.27) | 16.27 (13.12) | BLLQ | 4.66 (45.26) |
| 120 hr, | | | | |
| μg/sqcm/hr | | | | |
| (% RSD) | | | | |
| Avg Flux 120- | — | — | BLLQ | 2.20 (61.51) |
| 144 hr, | | | | |
| μg/sqcm/hr | | | | |
| (% RSD) | | | | |

### Example 17 (Solution)

Synthetic cannabidiol (CBD) formulations for transdermal delivery ((Formulation Nos. 001, 002, 003, 004, and 005) were prepared by mixing ingredients as shown in Table 15:

TABLE 15
Transdermal Synthetic Cannabidiol formulations

| | 001 | 002 | 003 | 004 | 005 |
|---|---|---|---|---|---|
| Ingredients | (% W/W) | (% W/W) | (% W/W) | (% W/W) | (% W/W) |
| CBD | 9.35 | 9.06 | 9.34 | 9.09 | 9.13 |
| PG | 90.65 | 45.51 | 45.17 | 45.54 | 45.33 |
| Hexylene | | 45.43 | | | |
| Glycol | | | | | |
| 1,3 Butanediol | | | 45.49 | | |
| PEG-400 | | | | 45.37 | |
| Dipropylene | | | | | 45.53 |
| Glycol | | | | | |

Abbreviations:
PG = propylene glycol;
CBD = Cannabidiol;
PEG-400: Polyethylene Glycol-400.

All of the components from Table 15, with the exception of the CBD, were mixed together with stirring for 18 hours. Next, the CBD was added into the excipient mixture to prepare the final transdermal formulations.

The prepared transdermal formulations were then subjected to a flux measurement test as follows. Human cadaver skin, stored at −80° C., was thawed at room temperature in phosphate buffered saline (PBS), and visually inspected for defects before using in the study. Transdermal flux was then measured using standard Franz diffusion cells composed of a cylindrical donor compartment and a separate water jacketed cylindrical receptor compartment with the volume of 13 mL. The human cadaver skin was clamped between the two compartments with the dermis side facing toward the receptor compartment. The donor compartment was filled with the transdermal CBD formulations prepared as described above. The receptor compartment was filled with receptor medium, held at constant temperature, and constantly stirred to collect the CBD as it diffuses through the skin and into receptor compartment. It is important to confirm that the receptor fluid is always in contact with the skin. The receptor compartment was emptied at 24 hr intervals for assay of CBD and replaced with fresh receptor solution. In order to maintain the sink condition in receptor compartment, it is important to keep the CBD concentration in receptor compartment less than 10% of its solubility. The experimental conditions are provided in Table 5:

TABLE 5
Experimental Condition for In-vitro Permeability testing

| | De-ionized water + 0.5% Brij-O(20) + |
|---|---|
| Receiving Media | 0.01% Sodium Azide |
| Receiving Media Volume (mL) | 13 |
| Sample Volume (mL) | 13 |
| Sampling Interval (hr) | 24, 48, 72, 96, 120, 144 |
| Franz-cell diffusion area (sqcm) | 1.76 |
| Membrane Type | Human Cadaver Skin |
| Mixing Speed | 600 RPM |

Flux of CBD through the human cadaver skin was measured for a minimum period of 144 Hrs (6 days) and results of the flux measurement are provided in Table 16.

TABLE 16
CBD Flux Results

| | 001 | 002 | 003 | 004 | 005 |
|---|---|---|---|---|---|
| Total Amount of CBD | 85795 | 167045 | 150000 | 59091 | 166477 |
| Permeated at 144 hrs | | | | | |
| (ng/cm²) | | | | | |
| Flux (ng/cm²/hr) | 338.5 | 1160.03 | 1041.66 | 410.35 | 1156.09 |

### Example 18 (Solution)

Additional synthetic Cannabidiol (CBD) formulations for transdermal delivery (Formulation Nos. 006 through 014) were prepared by mixing ingredients as shown in Table 17:

TABLE 17
Transdermal Synthetic Cannabidiol formulation no. 015 to 022

| | 015 (% | 016 (% | 017 (% | 018 (% | 019 (% | 020 (% | 021 (% | 022 (% |
|---|---|---|---|---|---|---|---|---|
| Ingredients | W/W) | W/W) | W/W) | W/W) | W/W) | W/W) | W/W) | W/W) |
| CBD | 9.95 | 9.64 | 9.77 | 9.98 | 9.98 | 9.64 | 9.87 | 9.52 |
| PG | 42.70 | 42.58 | 42.51 | 42.51 | 42.02 | 42.45 | 42.47 | 42.34 |
| 1,3 Butanediol | 42.36 | 42.48 | 42.40 | 42.63 | 42.66 | 42.77 | 42.50 | 42.64 |
| Tween-20 | 4.99 | | | | | | | |
| Triacetin | | 5.30 | | | | | | |
| PGML | | | 5.32 | | | | | |
| OA | | | | 4.88 | | | | |
| ML | | | | | 5.34 | | | |
| IPM | | | | | | 5.16 | | |
| IPP | | | | | | | 5.04 | |
| Labrasol | | | | | | | | 4.91 |

Abbreviations:
CBD = Cannabidiol;
PGML: Propylene glycol monolaurate;
PG = propylene glycol;
OA = Oleyl Alcohol;
ML = Methyl Laurate;
IPM = Isopropyl Myristate;
IPP: Isopropyl Palmitate.

Synthetic Cannabidiol formulations for transdermal delivery (006-014) were prepared by the same procedure described in Example 6. Flux measurement was also performed as described in Example 17. The experimental conditions are the same as provided in Table 5 of Example 17.

Flux of CBD through the human cadaver skin was measured for a minimum period of 48 Hrs and results of the flux measurement experiments are provided in Table 18.

TABLE 18
CBD Flux Results

| | Formulation No. | | | | | | | |
|---|---|---|---|---|---|---|---|---|
| | 015 | 016 | 017 | 018 | 019 | 020 | 021 | 022 |
| Total Amount | 25791 | 22851 | 29098 | 37085 | 37351 | 45008 | 59954 | 21524 |
| of CBD | | | | | | | | |
| Permeated at | | | | | | | | |
| 48 hrs | | | | | | | | |
| (ng/cm²) | | | | | | | | |
| Flux | 537.31 | 476.06 | 606.20 | 772.60 | 778.14 | 937.67 | 1249.04 | 448.41 |
| (ng/cm²/hr) | | | | | | | | |

### Example 19 (Solution)

Additional synthetic Cannabidiol (CBD) formulations for transdermal delivery (Formulation Nos. 160,162,175,176,177) were prepared by mixing ingredients as shown in Table 19:

TABLE 19
Transdermal Synthetic Cannabidiol formulation no. 160 to 177

| | 160 | 162 | 175 | 176 | 177 |
|---|---|---|---|---|---|
| Ingredients | (% W/W) | (% W/W) | (% W/W) | (% W/W) | (% W/W) |
| CBD | 9.93% | 9.97% | 9.90% | 9.93% | 9.93% |
| PG | 40.07% | 59.80% | 39.27% | 50.33% | 19.87% |
| ethanol | 50.00% | 19.93% | 20.46% | 19.87% | 50.00% |
| Water | | 10.30% | 10.56% | 9.93% | |
| DMSO | | | 19.80% | | 20.20% |
| IPP | | | | 9.93% | |

Abbreviations:
CBD = Cannabidiol;
DMSO: Dimethyl Sulfoxide
PG = propylene glycol;
IPP: Isopropyl Palmitate.

Synthetic Cannabidiol formulations for transdermal delivery (160-177) were prepared by the same procedure described in Example 17. Flux measurement was also performed as described in Example 17. The experimental conditions are the same as provided in Table 5 of Example 17.

Flux of CBD through the human cadaver skin was measured for a minimum period of 120 Hrs and results of the flux measurement experiments are provided in Table 20

TABLE 20
CBD Flux Results

| Flux | | | | | |
|---|---|---|---|---|---|
| (ug/ | Formulation No. | | | | |
| sqcm/hr) | 160 | 162 | 175 | 176 | 177 |
| 0-24 Hr | 0.61 | 0.39 | 0.6 | 1.31 (22%) | 1.03 (41%) |
| | (87%) | (67%) | (51%) | | |
| 24-48 Hr | 1.49 | 1.37 | 1.36 | 1.21 (13%) | 1.41 (17%) |
| | (6.5%) | (13%) | (17%) | | |
| 48-72 Hr | 0.95 | 1.22 | 1.12 | 0.87 (20%) | 1.13 (20%) |
| | (71%) | (6.8%) | (13%) | | |
| 72-96 Hr | | 1.03 | 0.93 | 0.71 (12%) | 0.91 (28%) |
| | | (2%) | (14%) | | |
| 96-120 Hr | | 0.81 | 0.70 | 0.6 (20%) | 0.73 (16%) |
| | | (10%) | (14%) | | |

While the invention has been described in detail and with reference to specific examples thereof, it will be apparent to one skilled in the art that various changes and modifications can be made therein without departing from the spirit and scope thereof.

What is claimed is:

1. A pharmaceutical composition comprising at least one active agent selected from the group consisting of:
about 3% to about 15% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof;
about 3% to about 15% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof; and
combinations thereof,
further wherein the pharmaceutical composition comprises:
about 0% to about 97% of at least one solvent;
about 0% to about 97% of at least surfactant;
optionally, about 0% to about 97% of at least one permeation enhancer; and/or
optionally, about 0% to about 97% of an adhesive and/or polymer.

2. The pharmaceutical composition of claim 1 wherein the active agent is present at a concentration selected from the group consisting of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, and about 50% of the formulation.

3. The pharmaceutical composition of claim 1 wherein the active agent is present at a concentration selected from the group consisting of about 1% to about 10%, about 1% to about 9%, about 1b % to about 8%, about 1b % to about 7%, about 1b % to about 6%, about 1b % to about 5%, about 0.1 to about 50%, about 1 to 20%, of about 5% to 25%, about 10% to about 20%, or about 15% to about 18%, about 30% to about 70%, and about 35% to about 65% of the formulation.

4. The pharmaceutical composition of claim 1 wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof.

5. The pharmaceutical composition of claim 1 wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof.

6. The pharmaceutical composition of claim 1 wherein the pharmaceutical composition comprises one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery.

7. The pharmaceutical composition of claim 1 wherein the pharmaceutical composition is formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, a formulation in an infusion pump, or transdermal drug-in-adhesive matrix formulation.

8. The pharmaceutical composition of claim 1 wherein the pharmaceutical composition provides a continuous, sustained delivery of the pharmaceutical composition to mitigate peak and valley pharmacokinetic behavior of the active agent.

9. The pharmaceutical composition of claim 1 wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

10. The pharmaceutical composition of claim 1 wherein the pharmaceutical composition provides a continuous, sustained delivery of the pharmaceutical composition via intravenous or subcutaneous infusion.

11. The pharmaceutical composition of claim 1 wherein the pharmaceutical composition provides a continuous, sustained delivery of the pharmaceutical composition via subcutaneous infusion.

12. The pharmaceutical composition of claim 1 wherein the pharmaceutical composition provides a continuous, sustained delivery of the pharmaceutical composition via an infusion pump device.

13. The pharmaceutical composition of claim 1 wherein the pharmaceutical composition provides a continuous, sustained delivery of the pharmaceutical composition via an ambulatory/portable infusion pump that is worn by the patient and may use replaceable cartridges.

14. The pharmaceutical composition of claim 1 wherein the pharmaceutical composition provides a continuous, sustained delivery of the pharmaceutical composition via a patch pump or micro pump.

15. The pharmaceutical composition of claim 1 wherein the pharmaceutical composition is formulated as a topical liquid formulation, topical semisolid formulation, topical gel formulation, topical polymer matrix formulation, topical adhesive matrix formulation, topical film forming gel formulation, a formulation in an infusion pump, or topical film forming spray formulation.

16. The pharmaceutical composition of claim 1 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof.

17. The pharmaceutical composition of claim 1 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v.

18. The pharmaceutical composition of claim 1 wherein the pharmaceutical composition is formulated as a transdermal patch.

19. The pharmaceutical composition of claim 1 wherein the pharmaceutical composition is formulated as a multilayer transdermal matrix system, a metered dose transdermal gel, metered dose transdermal spray, a film forming gel, a film forming spray, or a meter-dose aerosol.

20. The pharmaceutical composition of claim 1 wherein the pharmaceutical composition is formulated as a topical patch.

21. The pharmaceutical composition of claim 1 wherein the pharmaceutical composition is formulated as metered dose gel, metered dose spray, gel, cream, solution, emulsion, liquid compositions, semisolid compositions, a matrix of adhesive in combination with polymers, or film forming formulations.

22. The pharmaceutical composition of claim 1 wherein the pharmaceutical composition is formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof.

23. The pharmaceutical composition of claim 1 wherein the pharmaceutical composition is formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof.

24. The pharmaceutical composition of claim 1 wherein the pharmaceutical composition is formulated as a transdermal formulation which can be administered in a dosage regimen selected from the group consisting of once daily, twice daily, three times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

25. The pharmaceutical composition of claim 1 wherein the pharmaceutical composition is formulated as a topical formulation which can be administered in a dosage regimen selected from the group consisting of once daily, twice daily, three times a day, four times a day, five times a day, six times a day, once in 1-8 hrs, once in 1-24 hrs, once in two days, once in three days, once in four days, once in five days, once in six days, once in a week, once in a 8 to about 13 days, once in two weeks, and once in 15 days to about 30 days.

26. The pharmaceutical composition of claim 1 wherein the pharmaceutical composition is formulated as microneedles.

27. The pharmaceutical composition of claim 1 wherein the said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route.

28. The pharmaceutical composition of claim 1 indicated for the treatment and/or prevention and/or control of cancer in a patient, wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and ahematological cancer, such as multiple myeloma, B-cell lymphoma, T-cell lymphoma, Hodgkin's lymphoma/primary mediastinal B-cell lymphoma, and chronic myelogenous leukemia.

29. The pharmaceutical composition of claim 1 wherein said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route.

30. The pharmaceutical composition of claim 1 wherein said tetrahydrocannabinol (THC), cannabidiol (CBD), or derivative thereof is produced by a synthetic route.

31. The pharmaceutical composition of claim 1 wherein the active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition.

32. The pharmaceutical composition of claim 1 wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, and about 5 μg/mL.

33. The pharmaceutical composition of claim 1 co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

34. A method for the treatment and/or prevention and/or control of cancer in a patient comprising:
selecting a patient in need of treatment and/or prevention and/or control of cancer;
applying a pharmaceutical composition comprising at least one active agent selected from the group consisting of:
about 3% to about 15% of an active agent selected from the group consisting of cannabidiol (CBD), synthetic forms thereof, biosynthetic forms thereof, free base forms thereof, salts thereof, isomers thereof, amorphous forms thereof, derivatives thereof, and combinations thereof;
about 3% to about 15% of an active agent selected from the group consisting of tetrahydrocannabinol (THC), synthetic forms thereof, biosynthetic forms thereof, free base forms thereof, salts thereof, isomers thereof, amorphous forms thereof, derivatives thereof, and combinations thereof; and
combinations thereof,
further wherein the pharmaceutical composition comprises:
about 0% to about 97% of at least one solvent;
about 0% to about 97% of at least surfactant;
optionally, about 0% to about 97% of at least one permeation enhancer; and/or
optionally, about 0% to about 97% of an adhesive and/or polymer,
thereby treating and/or preventing and/or controlling cancer in the patient.

35. The method of claim 34, wherein the cancer is selected from the group consisting of endometrial cancer, breast cancer, ovarian cancer, cervical cancer, fallopian tube cancer, testicular cancer, primary peritoneal cancer, colon cancer, colorectal cancer, small intestine cancer, squamous cell carcinoma of the anogenital region, melanoma, renal cell carcinoma, lung cancer, non-small cell lung cancer, squamous cell carcinoma of the lung, stomach cancer, bladder cancer, gall bladder cancer, liver cancer, thyroid cancer, laryngeal cancer, salivary gland cancer, esophageal cancer, head and neck cancer, squamous cell carcinoma of the head and neck, prostate cancer, pancreatic cancer, mesothelioma, Merkel cell carcinoma, sarcoma, glioblastoma, GBM, and ahematological cancer, such as multiple myeloma, B-cell lymphoma, T-cell lymphoma, Hodgkin's lymphoma/primary mediastinal B-cell lymphoma, and chronic myelogenous leukemia.

36. The method of claim 34 wherein the cancer is glioblastoma, GBM.

37. The method of claim 34 further comprising the topical application of a transdermal pharmaceutical composition for the treatment and/or prevention and/or control of cancer in a patient, wherein the transdermal pharmaceutical composition is applied at a time period selected from the group consisting of once in a day, once in two days, once in three days, once in four days, once in five days, once in six days, once in a week, and once in ten days.

38. The method of claim 34 further providing a constant rate of delivery of the active components of the pharmaceutical composition over a time period.

39. The method of claim 34 further providing a steady absorption rate of the active components of the pharmaceutical composition over a time period.

40. The method of claim 34 further achieving a constant blood serum levels of the active component of the pharmaceutical composition over a time period.

41. The method of claim 34 further achieving a reduced variability in dosage of the active component of the pharmaceutical composition over a time period.

42. The method of claim 34 further providing a plasma concentration of the active component of the pharmaceutical composition in a therapeutic range over a period of time.

43. The method of claim 34 further providing a plasma concentration of the active component of the pharmaceutical composition in a therapeutic range of about 0.1 ng/mL to about 500 ng/mL.

44. The method of claim 34 further providing a continuous delivery system which provides a continuous, sustained delivery of the pharmaceutical composition to the patient via intravenous or subcutaneous infusion.

45. The method of claim 34 further providing a continuous delivery system which provides a continuous, sustained delivery of the pharmaceutical composition to the patient via subcutaneous infusion.

46. The method of claim 34 further providing a continuous delivery system which provides a continuous, sustained delivery of the pharmaceutical composition to the patient via an infusion pump device.

47. The method of claim 34 further providing a continuous delivery system which provides a continuous, sustained delivery of the pharmaceutical composition to the patient via an ambulatory/portable infusion pump that is worn by the patient and may use replaceable cartridges.

48. The method of claim 34 further providing a continuous delivery system which provides a continuous, sustained delivery of the pharmaceutical composition to the patient via a patch pump or micro pump.

49. The method of claim 34 further providing a continuous delivery system which comprises a pharmaceutical composition selected from the group consisting of a liquid formulation, a solid formulation, a semi-solid formulation, an emulsion formulation, a nanoparticle formulation, a matrix formulation, a film formulation, a patch formulation, a formulation in an infusion pump, and/or combinations thereof.

50. The method of claim 34 wherein the pharmaceutical composition is in a dosage form selected from the group consisting of: cream, lotion, gel, oil, ointment, suppository, spray, foam, liniment, aerosol, buccal and sublingual tablet, a transdermal device, and a transdermal patch.

51. The method of claim 34 wherein the pharmaceutical composition is co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.

52. The method of claim 34 wherein the active agent is present at a concentration selected from the group consisting of about 1%, about 1.5%, about 2%, about 2.5%, about 3%, about 3.5%, about 4%, about 4.5%, about 5%, about 5.5%, about 6%, about 6.5%, about 7%, about 7.5%, about 8%, about 8.5%, about 9%, about 9.5%, about 10%, about 15%, about 20%, about 25%, about 30%, about 35%, about 40%, about 45%, and about 50% of the formulation.

53. The method of claim 34 wherein the active agent is present at a concentration selected from the group consisting of about 1% to about 10%, about 1% to about 9%, about 1% to about 8%, about 1% to about 7%, about 1% to about 6%, about 1% to about 5%, about 0.1 to about 50%, about 1 to 20%, of about 5% to 25%, about 10% to about 20%, or about 15% to about 18%, about 30% to about 70%, and about 35% to about 65% of the formulation.

54. The method of claim 34 wherein the THC is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, stereoisomers thereof, solid solution thereof, ion-pair thereof, solution thereof, powder form thereof, liquid form thereof, alone or combinations thereof.

55. The method of claim 34 wherein the CBD is selected from the group comprising of free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, ion-pairs thereof, stereoisomers thereof, solid solution thereof, solution thereof, powder form thereof, liquid form thereof, alone or combinations thereof.

56. The method of claim 34 wherein the pharmaceutical composition comprises one or more active agent selected from the group consisting of tetrahydrocannabinol (THC), cannabidiol (CBD), the free base thereof, salts thereof, isomers thereof, amorphous forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, polymorph thereof, solid solution thereof, coated form thereof, and combinations thereof, in a dosage form for transdermal delivery.

57. The method of claim 34 wherein the pharmaceutical composition is formulated as transdermal liquid formulation, transdermal semisolid formulation, transdermal gel formulation, or transdermal polymer matrix formulation, transdermal adhesive matrix formulation, transdermal film forming gel, transdermal film forming spray formulation, multilayer transdermal matrix system, a formulation in an infusion pump, or transdermal drug-in-adhesive matrix formulation.

58. The method of claim 34 wherein the pharmaceutical composition provides a continuous, sustained delivery of the pharmaceutical composition to mitigate peak and valley pharmacokinetic behavior of the active agent.

59. The method of claim 34 wherein the pharmaceutical composition provides a continuous, sustained delivery of the pharmaceutical composition via administration to the patient by a route selected from the group consisting of parenteral, intravenous, subcutaneous, intramuscular, intrathecal, oral, buccal, mucosal, intranasal, rectal, vaginal, transdermal, implantable, topical, and combinations thereof.

60. The method of claim 34 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, tackifier, diluent, bulking agent, solubilizers, suspending agents, dispersing agents, stabilizers, plasticizers, surfactants, antioxidants, oxidants, and combinations thereof.

61. The method of claim 34 further comprising carriers or ingredients in effective amount selected from the group consisting of solvents, gelling agents, polymers, pressure sensitive adhesive polymers, penetration enhancers, emollients, skin irritation reducing agents, buffering agents, pH stabilizers, solubilizers, suspending agents, dispersing agents, stabilizers, plasticizers, tackifiers, diluents, bulking agents, surfactants, antioxidants, oxidants, and combinations thereof in the range of 0.1%-99.5% w/w or w/v.

62. The method of claim 34 wherein the pharmaceutical composition is formulated as a transdermal patch.

63. The method of claim 34 wherein the pharmaceutical composition is formulated as a multilayer transdermal matrix system, a metered dose transdermal gel, metered dose transdermal spray, a film forming gel, a film forming spray, or a meter-dose aerosol.

64. The method of claim 34 wherein the pharmaceutical composition is formulated as a topical patch.

65. The method of claim 34 wherein the pharmaceutical composition is formulated as metered dose gel, metered dose spray, gel, cream, solution, emulsion, liquid compositions, semisolid compositions, a matrix of adhesive in combination with polymers, or film forming formulations.

66. The method of claim 34 wherein the pharmaceutical composition is formulated as a transdermal patch, wherein the transdermal patch is selected from the group such as to reservoir patch, a multilayer transdermal matrix system, a microreservoir patch, a matrix patch, a drug in adhesive patch, a matrix patch of adhesive in combination with polymers, a pressure sensitive adhesive patch, extended-release transdermal film a liquid reservoir system, a microreservoir patch, a mucoadhesive patch, and combinations thereof.

67. The method of claim 34 wherein the pharmaceutical composition is formulated as a topical patch, wherein the topical patch is selected from the group such as a multilayer transdermal matrix system, reservoir patch, a microreservoir patch, a matrix patch, a drug in adhesive patch, a pressure sensitive adhesive patch, extended-release transdermal film a liquid reservoir system, a microreservoir patch, a mucoadhesive patch, a micro-dosing patch, a matrix patch of adhesive in combination with polymers, and combinations thereof.

68. The method of claim 34 wherein the pharmaceutical composition is formulated as microneedles.

69. The method of claim 34 wherein the said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, naturally occurring forms thereof, active metabolites thereof, and combinations thereof is produced by a synthetic route.

70. The method of claim 34 wherein said of tetrahydrocannabinol (THC), cannabidiol (CBD), the free base thereof, salts thereof, isomers thereof, amorphous forms thereof, polymorphs forms thereof, stereoisomers thereof, ion-pairs thereof, coated forms thereof, crystalline forms thereof, co-crystalline forms thereof, prodrugs thereof, analogs thereof, derivatives thereof, synthetic forms thereof, biosynthetic forms thereof, active metabolites thereof, and combinations thereof is produced by a synthetic route.

71. The method of claim 34 wherein said tetrahydrocannabinol (THC), cannabidiol (CBD), or derivative thereof is produced by a synthetic route.

72. The method of claim 34 wherein the active agent is produced synthetically and has a purity equal to or greater than about 90%, 91%, 92%, 93%, 94%, 95%, 96%, 97%, 98%, 99%, 99.5%, 99.75%, or 100% (w/w) before being added to said pharmaceutical composition.

73. The method of claim 34 wherein the pharmaceutical composition provides a blood serum level of active agent selected from the group consisting of about 0.01 ng/mL, about 0.02 ng/mL, about 0.05 ng/mL, about 0.1 ng/mL, about 0.2 ng/mL, about 0.5 ng/mL, about 1 ng/mL, about 2 ng/mL, about 5 ng/mL, about 10 ng/mL, about 20 ng/mL, about 50 ng/mL, about 100 ng/mL, about 200 ng/mL, about 500 ng/mL, about 1 μg/mL mL, about 2 μg/mL, and about 5 μg/mL.

74. The method of claim 34 wherein the pharmaceutical composition is co-administered with at least one additional therapeutic selected from the group consisting of: temozolomide, peptides, polypeptides, fusion proteins, nucleic acid molecules, small molecules, mimetic agents, synthetic drugs, inorganic molecules, organic molecules, chemotherapies, radiation therapies, hormonal therapies, anti-angiogenesis therapies, targeted therapies, and/or biological therapies including immunotherapies and surgery, and combinations thereof.
